SEQUENCE LISTING (9 sequences)

SEQ ID NO: 1 - RNA, 22 nt, synthetic construct
  misc_feature 1..22: note = Description of Artificial Sequence: Synthetic oligonucleotide
  source 1..22: mol_type = other RNA
tggaatgtaaagaagtatgtat

SEQ ID NO: 2 - RNA, 22 nt, synthetic construct
  misc_feature 1..22: note = Description of Artificial Sequence: Synthetic oligonucleotide
  source 1..22: mol_type = other RNA
tttggtccccttcaaccagctg

SEQ ID NO: 3 - RNA, 22 nt, synthetic construct
  misc_feature 1..22: note = Description of Artificial Sequence: Synthetic oligonucleotide
  source 1..22: mol_type = other RNA
ataagacgagcaaaaagcttgt

SEQ ID NO: 4 - RNA, 21 nt, synthetic construct
  misc_feature 1..21: note = Description of Artificial Sequence: Synthetic oligonucleotide
  source 1..21: mol_type = other RNA
ttaagacttgcagtgatgttt

SEQ ID NO: 5 - DNA, 25 bp, synthetic construct
  misc_feature 1..25: note = Description of Artificial Sequence: Synthetic oligonucleotide
  source 1..25: mol_type = other DNA
caccgtgtcgtaccacccaggacga

SEQ ID NO: 6 - DNA, 22 bp, synthetic construct
  misc_feature 1..22: note = Description of Artificial Sequence: Synthetic oligonucleotide
  source 1..22: mol_type = other DNA
aaactcgtcctgggtacgacac

SEQ ID NO: 7 - DNA, 25 bp, synthetic construct
  misc_feature 1..25: note = Description of Artificial Sequence: Synthetic oligonucleotide
  source 1..25: mol_type = other DNA
caccggtcttggttcttagggccac

SEQ ID NO: 8 - DNA, 25 bp, synthetic construct
  misc_feature 1..25: note = Description of Artificial Sequence: Synthetic oligonucleotide
  source 1..25: mol_type = other DNA
caccggtcttggttcttagggccac

SEQ ID NO: 9 - protein, 355 aa, Homo sapiens
MRGVRAATAAAVAATAASGLSRREAGGRAGAAAAVVRPPGRKCGRCRRLANFPGRKRRRRRRKGLGATTGGCGEAVSSLHPAPHSPSSVRPAGRRARRQRRGAGSAERPMSGAGEALAPGPVGPQRVAEAGGGQLGSTAQGKCDKDNTEKDITQATNSHFTHGEMQDQSIWGNPSDGELIRTQPQRLPQLQTSAQVPSGEEIGKIKNGHTGLSNGNGIHHGAKHGSADNRKLSAPVSQKMHRKIQSSLSVNSDISKKSKVNAVFSQKTGSSPEDCCVHCILACLFCEFLTLCNIVLGQASCGICTSEACCCCCGDEMGDDCNCPCDMDCGIMDACCESSDCLEICMECCGICFPS

(19) United States
(12) Patent Application Publication
(10) Pub. No.: US 2024/0197640 A1
(43) Pub. Date: Jun. 20, 2024

(54) SYSTEMS AND METHODS FOR EXOSOME DELIVERY OF MICRORNAS FOR CELLULAR REPROGRAMMING

(71) Applicant: Duke University, Durham, NC (US)

(72) Inventors: Conrad P. Hodgkinson, Durham, NC (US); Hualing Sun, Durham, NC (US); Victor J. Dzau, Durham, NC (US)

(21) Appl. No.: 18/474,697

(22) Filed: Sep. 26, 2023

Related U.S. Application Data
(63) Continuation of application No. PCT/US22/21903, filed on Mar. 25, 2022.
(60) Provisional application No. 63/166,356, filed on Mar. 26, 2021.
(60) Provisional application No. 63/278,769, filed on Nov. 12, 2021.

(51) Int. Cl. A61K 9/50; A61P 9/00; C12N 5/071; C12N 15/113

(52) U.S. Cl. CPC A61K 9/5068; A61P 9/00; C12N 5/069; C12N 15/113; C12N 2310/141

(57) ABSTRACT

The present disclosure describes, in part, exosomes comprising miRNA for the reprogramming of fibroblasts and methods of using the same.

### RELATED APPLICATIONS

This application is a continuation of International Patent Application No. PCT/US2022/021903, filed Mar. 25, 2022, which claims the benefit of priority under 35 U.S.C. § 119(e) to U.S. Provisional Application No. 63/166,356, filed Mar. 26, 2021, and Provisional Application No. 63/278,769, filed Nov. 12, 2021, the entire contents of which are incorporated herein by reference in their entireties.

### GOVERNMENT INTEREST

This invention was made with government support under (grant R01 HL131814-01A1 awarded by the National Heart, Lung, and Blood Institute. The government has certain rights in the invention.

### TECHNICAL FIELD

The current subject matter relates to the field of cardiology and repair of cardiac tissue after injury.

### SEQUENCE LISTING

This application contains a Sequence Listing which has been submitted electronically as a WIPO Standard ST.26 XML file via Patent Center, created on Sep. 26, 2023, is entitled “123658-10403.xml” and is 12.4 KB in size. The sequence listing is incorporated herein by reference in its entirety.

### BACKGROUND

Directly reprogramming scar fibroblasts into new cardiomyocytes improves cardiac function in the infarcted heart. However, a major challenge is the delivery of reprogramming factors into the cardiac tissue. Currently, reprogramming factors are delivered into the heart by viruses. Virus delivery is associated with a number of drawbacks such as a lack cell specificity and packaging size constraints.

### SUMMARY

The invention provides a solution to the drawbacks associated with virus delivery. Accordingly, the invention features a loaded exosome comprising an exosome isolated from an endothelial cell and at least one exogenous miR comprised within the exosome. In one example, the exosome is derived from a C166 cell (American Type Culture Collection (ATCC) under the cell line designation. CRL 2581™). Formation of cardiac fibroblasts is a complex process involving precursors or endothelial cells undergoing endothelial to mesenchymal or epithelial to mesenchymal transitions depending upon context. Thus, such cells may also be used as sources of exosomes for the delivery and/or therapeutic methods described herein.

An exemplary exosome comprises miR148a-3p. In another example, at least one exogenous miR is selected from the group consisting of miR1, miR126, miR133, miR138, mir148a-3p, miR206, miR208, miR499-5p and any combinations thereof.

Additional examples are described as follows:
the loaded exosome comprises 2 exogenous miR selected from the group consisting of miR1, miR126, miR133, miR138, miR206, miR208, and miR499-5p;
the loaded exosome comprises 3 exogenous miR selected from the group consisting of miR1, miR126, miR133, miR138, miR206, miR208, and miR499-5p;
the loaded exosome comprises 4 exogenous miR selected from the group consisting of miR1, miR126, miR133, miR138, miR206, miR208, and miR499-5p;
the loaded exosome comprises more than 4 exogenous miR selected from the group consisting of miR1, miR126, miR133, miR138, miR206, miR208, and miR499-5p; and/or the loaded exosome of any one of the exosomes described above, comprising miR1, miR133, miR208 and miR499-5p.

A loaded exosome comprising an exosome isolated from a mammalian cell and at least one exogenous miR comprised within the exosome, wherein the at least one exogenous miR comprises miR148a-3p is encompassed by the invention. Additional examples are described as follows:
the loaded exosome, further comprising at least one miR selected from the group consisting of miR1, miR126, miR133, miR138, miR206, miR208, miR499-5p and any combinations thereof;
the loaded exosome, further comprising 2 miRs selected from the group consisting of miR1, miR126, miR133, miR138, miR206, miR208, and miR499-5p;
the loaded exosome, further comprising 3 miRs selected from the group consisting of miR1, miR126, miR133, miR138, miR206, miR208, and miR499-5p;
the loaded exosome, further comprising 4 miRs selected from the group consisting of miR1, miR126, miR133, miR138, miR206, miR208, and miR499-5p;
the loaded exosome, further comprising more than 4 miRs selected from the group consisting of miR1, miR126, miR133, miR138, miR206, miR208, and miR499-5p; or the loaded exosome comprising miR1, miR133, miR208 and miR499-5p.

Also within the invention is an engineered cell for producing exosomes, comprising a mammalian endothelial cell and a cassette for expression of one or more exogenous miR. For example, the engineered cell comprises a C166 cell or any endothelial cell, epithelial cell, or other cell described herein such as those available from ATCC, Promocell, or as described by Rahman N A, Raisal A N H M, Meyding-Lamade U, Craemer E M, Diah S, Tuah A A, and Muharram S H. Brain Research 1642 (2016) 532-545. For example, the engineered cell comprises miR148a-3p.

The invention encompasses an engineered cell for producing exosomes, comprising a mammalian cell, e.g., the cell comprises miR148a-3p and one or more exogenous miR. For example, the engineered cell is one in which the at least one exogenous miR is selected from the group consisting of miR1, miR126, miR133, miR138, mir148a-3p, miR206, miR208, miR499-5p and any combinations thereof. Exemplary combinations are described below:
the engineered cell, comprising 2 exogenous miR selected from the group consisting of miR1, miR126, miR133, miR138, miR206, miR208, and miR499-5p;
the engineered cell, comprising 3 exogenous miR selected from the group consisting of miR1, miR126, miR133, miR138, miR206, miR208, and miR499-5p;
the engineered cell, comprising 4 exogenous miR selected from the group consisting of miR1, miR126, miR133, miR138, miR206, miR208, and miR499-5p;
the engineered cell, comprising more than 4 exogenous miR selected from the group consisting of miR1, miR126, miR133, miR138, miR206, miR208, and miR499-5p; or
the engineered cell of claim 21 comprising miR1, miR133, miR208 and miR499-5p.

A system for cell delivery comprising a loaded exosome and an inhibitor of MDFIC (Myo D Family Inhibitor Domain Containing) protein expression is also within the scope of the invention. For example, the inhibitor of MDFIC expression comprises a nucleic acid. In an example, the system may encompass a nucleic acid which is selected from the group consisting of miR, siRNA, and shRNA. For example, the nucleic acid comprises miR148a-3p. Alternatively, the nucleic acid comprises an siRNA or shRNA directed against MDFIC. Exemplary systems are characterized by one or more of the following features:
wherein the loaded exosome of the system comprises at least one exogenous miR;
wherein the at least one exogenous miR is selected from the group consisting of miR1, miR126, miR133, miR138, mir148a-3p, miR206, miR208, miR499-5p and any combinations thereof;
comprising 2 exogenous miR selected from the group consisting of miR1, miR126, miR133, miR138, miR206, miR208, and miR499-5p;
comprising 3 exogenous miR selected from the group consisting of miR1, miR126, miR133, miR138, miR206, miR208, and miR499-5p;
comprising 4 exogenous miR selected from the group consisting of miR1, miR126, miR133, miR138, miR206, miR208, and miR499-5p;
comprising more than 4 exogenous miR selected from the group consisting of miR1, miR126, miR133, miR138, miR206, miR208, and miR499-5p; or comprising miR1, miR133, miR208 and miR499-5p.

A method of reprogramming a cell comprises administering any one of the loaded exosomes described above. In another aspect, a method of reprogramming a cell comprises any one of the exemplar systems described above.

A method for manufacturing a loaded exosome comprises the follow steps:
providing an endothelial cell comprising an inhibitor of MDFIC expression and at least one exogenous miR; inducing the cell to produce loaded exosomes comprising the at least one exogenous miR; and harvesting the loaded exosome. An exemplary method utilizes an endothelial cell such as a C166 cell. In an exemplary method, the inhibitor of MDFIC expression comprises miR148a-3p. Further exemplary manufacturing methods are characterized by the following features:
wherein the at least one exogenous is selected from the group consisting of miR1, miR126, miR133, miR138, mir148a-3p, miR206, miR208, miR499-5p and any combinations thereof;
method comprising 2 exogenous miR selected from the group consisting of miR1, miR126, miR133, miR138, miR206, miR208, and miR499-5p;
method comprising 3 exogenous miR selected from the group consisting of miR1, miR126, miR133, miR138, miR206, miR208, and miR499-5p;
method comprising 4 exogenous miR selected from the group consisting of miR1, miR126, miR133, miR138, miR206, miR208, and miR499-5p;
method comprising more than 4 exogenous miR selected from the group consisting of miR1, miR126, miR133, miR138, miR206, miR208, and miR499-5p; or
method comprising miR1, miR133, miR208 and miR499-5p.

The invention encompasses a loaded exosome manufactured by any one of the methods described above.

A method of treating a cardiac disorder is carried out by delivering a loaded exosome characterized as described above and manufactured by any one of the methods described above. For example, the treatment or therapeutic protocol includes delivery of the loaded exosome is delivered to a cardiac fibroblast. For example, the loaded exosome is delivered to a cardiac tissue in vivo or the loaded exosome is delivered to myocardium tissue of a mammal. The animal to be treated in a mammal, e.g., a human subject or a veterinary subject. A veterinary subject includes a companion animal such as a dog or cat or a performance or working animal such as a horse or livestock animal, such as a pig.

The invention further encompasses a method of reducing cardiac fibrosis comprising delivering a loaded exosome, e.g., any one of the loaded exosomes described above to cardiac tissue in a mammal. The delivery results in the appearance of or increase in the number of cardiomyocytes in the cardiac tissue. A method for improving cardiac function comprises delivering a loaded exosome, e.g., any one of the loaded exosomes described above, to cardiac tissue of a mammal.

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The efficacy of direct cardiac reprogramming is improved by optimizing delivery as described and claimed herein. A fibroblast-targeting exosome was made and shown to the problems and challenges associated with previous microRNA (miR) delivery methods. Through a screening-based approach, C166-derived exosomes were found to be effective in targeting fibroblasts both in vitro and in vivo. C166-derived exosomes were similarly effective at delivering reprogramming factors. In vitro, reprogramming factors delivered by C166-derived exosomes induced both cardiomyocyte-specific gene expression and cardiomyocyte formation. In vivo, reprogramming factors delivered by C166-derived exosomes efficiently converted ˜20% of cardiac fibroblasts in the infarct border zone into cardiomyocytes. Reprogramming using the exosome-containing miRNAs was associated with significant improvements in cardiac function following myocardial infarction. The effects of C166 exosome mediated delivery of reprogramming factors were mediated miR-148a-3p. The target of miR-148a-3p was found to be MDFIC and knockdown of this protein enhanced reprogramming efficacy.

C166-derived exosomes are an effective method for delivering reprogramming factors to cardiac fibroblasts and miR-148a-3p is an example of miRNA, which enhances reprogramming efficacy.

The present disclosure is based, in part, on the discovery and development by the inventors of a fibroblast-specific delivery exosome for cardiac reprogramming factors. The delivery of reprogramming factors, specifically into fibroblasts, enhances the therapeutic outcomes of cardiac reprogramming in vivo.

Accordingly, one aspect of the present disclosure provides a loaded exosome comprising, consisting of, or consisting essentially of an exosome having located inside at least one miRNA. In some embodiments, the exosome is isolated from a cell selected from the group consisting of C3H10T1/2 (C3H), C166, macrophage (md), and combinations thereof. In another embodiment, the miRNA is selected from the group consisting mir1; mir133; mir138; mir206; mir208; mir126; mir1, mir133; mir1, mir138; mir1, mir206; mir1, mir208; mir133, mir138; mir133, mir206; mir133, mir208; mir138, mir206; mir138, mir208; mir206, mir208; mir1, mir138, mir208; mir1, mir206, mir208; mir138, mir206, mir208; mir1, mir133, mir206; mir1, mir133, mir208; mir1, mir138, mir206; mir133, mir138, mir208; and mir133, mir138, mir206; mir1, mir133, mir208, mir499-5p; mir1, mir133, mir206, mir499-5p; and combinations thereof.

Another aspect provides a pharmaceutical composition comprising a loaded exosome according to the present disclosure and a pharmaceutically acceptable diluent, excipient, or carrier.

Another aspect of the present disclosure provides a method for promoting the direct reprogramming of a cell into cardiomyocytes or cardiac tissue, the method comprising, consisting of, or consisting essentially of contact the cell with a loaded exosome as provided herein.

In some embodiments, the cell comprises cardiac fibrotic tissue. In addition to cardiac fibroblasts/cardiac fibrotic tissue, exosomal delivery is useful to deliver agents to mediate direct reprogramming other cells or tissues of a fibrotic phenotype, e.g., lung fibroblasts, liver fibroblasts, or kidney fibroblasts. For example, lung scarring/fibrosis is caused by infectious diseases such as viruses or bacteria, e.g., COVID-19, pneumonia, or exposure to damaging substances such as tobacco or asbestos. Kidney scarring is caused by diabetes, autoimmune disease and high blood pressure, regular use of certain medications and prolonged infections. Liver scarring/fibrosis is caused by many forms of liver diseases and conditions, such as hepatitis and chronic alcoholism. For example, chronic viral hepatitis (hepatitis B, C and D), fat accumulating in the liver (nonalcoholic fatty liver disease), iron buildup in the body (hemochromatosis), cystic fibrosis, copper accumulated in the liver (Wilson's disease), poorly formed bile ducts (biliary atresia), alpha-1 antitrypsin deficiency, inherited disorders of sugar metabolism (galactosemia or glycogen storage disease), genetic digestive disorder (Alagille syndrome), autoimmune hepatitis, primary biliary cirrhosis, primary sclerosing cholangitis, infection, such as syphilis or brucellosis, or medications, including methotrexate or isoniazid. Scarring (fibrosis) in brain tissue is caused by stroke, vascular injury, or impaired supply of blood to the brain as well as dementia, multiple sclerosis (MS), lupus, cancer, physical injury/trauma to brain tissue, or exposure to toxins such as heavy metals (lead, mercury, cadmium), certain drugs such as mefloquine (Lariam), or food additives.

Another aspect of the present disclosure provides a method of restoring tissue function to fibrotic tissue in an organ, the method comprising, consisting of, or consisting essentially of providing patient-derived fibroblasts and introducing to the fibroblasts a loaded exosome as provided herein.

In some embodiments, the patient-derived fibroblast comprises dermal fibroblasts. In other embodiments, the patient-derived fibroblasts comprise cardiac fibroblasts.

In one embodiment, the loaded exosomes are introduced ex vivo. In another embodiment, the loaded exosome are introduced in situ. In yet other embodiments, the loaded exosomes are introduced in vivo.

Another aspect of the present disclosure provides a method of preventing and/or treating an ischemic or reperfusion-related injury in a subject, the method comprising, consisting of, or consisting essentially of administering to the subject at risk of, or suffering from, the ischemic or reperfusion-related injury a therapeutically effective amount of a loaded exosome, or a pharmaceutical composition thereof, as provided herein.

In some embodiments, the subject is suffering from a cardiac disorder.

Another aspect of the present disclosure provides all that is described and illustrated herein.

Polynucleotides, polypeptides, or other agents described herein are preferably purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or nucleic acid sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A small molecule is a compound that is less than 2000 Daltons in mass. The molecular mass of the small molecule is preferably less than 1000 Daltons, more preferably less than 600 Daltons, e.g., the compound is less than 500 Daltons, 400 Daltons, 300 Daltons, 200 Daltons, or 100 Daltons.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF DRAWINGS

FIG. 1A is a series of photographs of cardiac fibroblasts. Exosomes were isolated from 1 million cells (C166 cells, C3H/10T1/2 cells, or macrophages) and transfected with a Texas-Red labeled siRNA. Once transfected with the Texas-Red labeled siRNA, exosomes were incubated with cardiac fibroblasts for 24 hours. After 24 hours, the cells were washed copiously and cultured in standard growth media. Imaging for Texas-Red labeling was carried out 48 hours after the removal of the labeled exosomes. N=3. Representative images shown. Scale bar 50 microns

FIG. 1B is a series of bar graphs showing the results of an evaluation of delivery vehicles. Exosomes were isolated from 1 million cells (C166 cells, C3H/10T1/2 cells, or macrophages) and transfected with either miR combo or the non-targeting miR, negmiR. NegmiR was used for bars 1, 3, 4 and 5. miR combo for bars 2, 6, 7 and 8. Once the exosomes were loaded with miR combo or negmiR they were incubated with cardiac fibroblasts for 24 hours. By way of a positive control, miR combo was also transfected into cardiac fibroblasts via the standard lipid based approach. After 24 hours, exosomes and transfection complexes were removed. Cardiomyocyte gene expression (Actn2 and Myh6) was assessed by qPCR 14-days after the removal of loaded exosomes and transfection complexes. Expression values were normalized to the housekeeping gene Gapdh and are expressed as a fold change compared to control cells. N=3 *P<0.05.

FIG. 1C is a bar graph and a photograph showing the number of reprogramming events following mIR delivery. Exosomes were isolated from 1 million C166 cells and transfected with either miR combo or the non-targeting miR negmiR. Once the exosomes were loaded with miR combo or negmiR they were incubated with cardiac fibroblasts for 24 hours. By way of a control, miR combo was also transfected into cardiac fibroblasts via the standard lipid carrier. After 24 hours, exosomes and transfection complexes were removed. Fourteen days after the removal of loaded exosomes and transfection complexes, cells were analyzed for the expression of the cardiomyocyte proteins Tnni3 (green) and Actn2 (red) by immunostaining. The number of cells expressing both proteins was determined. N=3 *P<0.05. Representative images shown. Scale bar 100 microns.

FIGS. 1A-1C collectively demonstrate that C166-derived exosomes are an efficient delivery vehicle for miR combo in vitro.

FIG. 2A is a bar graph and a photograph showing the results of an evaluation of delivery vehicles in vivo. Exosomes were isolated from 1-million C166 cells and transfected with FITC conjugated siRNA. Once transfected, the exosomes were injected into the border zone of a Fsp1-Cre:tdTomato mouse immediately after MI. One week later, cardiac tissue sections were analyzed for tdTomato expression (fibroblasts), Tnni3 (cardiomyocytes), Isolectin-B4 (endothelial cells) and FITC (internalized labeled exosomes). N=3. Representative images shown. Scale bar 100 microns. *** P<0.001 comparison between fibroblasts and cardiomyocytes. ###P<0.001 comparison between fibroblasts and endothelial cells. These data show that loaded exosomes target cardiac fibroblasts in vivo.

FIG. 2B is a bar graph and a photograph showing the results of an evaluation of cardiac repair and regeneration. Exosomes were isolated from 1-million C166 cells and transfected with either negmiR or miR combo. Following transfection, exosomes loaded with miRNAs were injected into the border zone of a Fsp1-Cre:tdTomato mouse immediately after MI. Two months later, serial cardiac tissue sections were analyzed for fibrosis. N=7. *** P<0.01. Representative serial sections from a single mouse heart are shown. Scale bar 1 mm.

FIG. 2C is a bar graph and a photograph showing the results of an evaluation of fibrosis in cardiac tissue. Exosomes were isolated from 1-million C166 cells and transfected with either negmiR or miR combo. Once transfected, the miRNA loaded exosomes were injected into the border zone of a Fsp1-Cre:tdTomato mouse immediately after MI. Two months later, serial cardiac tissue sections were analyzed for co-localization of tdTomato (red) and the cardiomyocyte marker Tnni3 (green). The number of tdTomato+cardiomyocytes (cardiomyocytes derived from the reprogramming of fibroblasts) are expressed as a percentage of the total cardiomyocyte population. N=7. *** P<0.001. Representative images are shown. Scale bar 100 microns.

FIG. 2D is a series of bar graphs showing the results of an evaluation of cardiac function. Exosomes were isolated from 1-million C166 cells and transfected with either negmiR or miR combo. Once transfected, the miR loaded exosomes, or an equivalent volume of PBS, were injected into the border zone of a Fsp1-Cre:tdTomato mouse immediately after MI. Cardiac function was determined by echocardiography at the indicated time-points. N=7. *** P<0.001 comparisons made to PBS control. ##P<0.01 comparisons made to negmiR control.

FIGS. 2A-D collectively demonstrate that C166-derived exosomes containing miR combo improves cardiac function in MI mice.

FIG. 3A is a diagram of the Drosha gene. Cas9 gene-editing was used to delete Drosha expression in C166 cells. Guide-RNAs were designed to exon 4 (the first coding exon) and exon 30.

FIG. 3B is a photograph of an electrophoretic gel showing Drosha expression. Drosha targeting guide-RNAs were cloned into a plasmid containing Cas9 and mCherry. Plasmids (Drosha gRNA-Cas9-mCherry or the control Cas9-mCherry) were transfected into C166 cells. One day after transfection, transfection complexes were removed and cells incubated for seven days in media containing blasticidin and puromycin. Surviving cells were isolated via flow sorting for the mCherry marker. Following amplification of the mCherry+ cells, Cas9 and Drosha expression was determined by immunoblotting. N=3. Representative images shown.

FIG. 3C is a series of bar graphs showing the results of an analysis of mRNA levels in cardiomyocytes. Exosomes from 1-million control (Cas9-mCherry) and Drosha knockout (Drosha gRNA-Cas9-mCherry) C166 cells were isolated and transfected with miR combo. Following transfection, miR combo loaded exosomes were incubated with cardiac fibroblasts for 24-hours. By way of a control, cardiac fibroblasts were also transfected with negmiR or miR combo via the standard lipid-based approach. After 24 hours, exosomes and transfection complexes were removed. Cardiomyocyte gene expression was assessed by qPCR 14-days after the removal of loaded exosomes and transfection complexes. Expression values were normalized to the housekeeping gene Gapdh and are expressed as a fold change to the control negmiR. N=3. *** P<0.001, ** P<0.01, *P<0.05 comparisons made to negmiR. #P<0.05 comparisons made to miR combo.

FIG. 3D is a series of bar graphs showing the results of an analysis of cardiac function. Exosomes from 1-million control (Cas9-mCherry) and Drosha knockout (Drosha gRNA-Cas9-mCherry) C166 cells were isolated and transfected with negmiR or miR combo. Once transfected, the miRNA loaded exosomes, or an equivalent volume of PBS, were injected into the border zone of C57BL6 mice immediately after MI. Cardiac function was determined by echocardiography at the indicated time-points. N=5. *** P<0.001 comparisons made to negmiR. ##P<0.01, #P<0.05 comparisons made between exosomes derived from control and Drosha knockout C166 cells. See Supplementary Table 2 for a full report of functional data.

FIGS. 3A-D collectively demonstrate that the effects of C166-derived exosomes are dependent on endogenous miRNAs.

FIG. 4A is a graph showing the results of an exosome miR profile analysis. miRNA-seq was performed on exosomes derived from C166 cells and macrophages. miRNAs which were found to be significantly higher in C166 cells are shown. Fold change is shown. N as indicated.

FIG. 4B is represents the read count for the indicated miRNAs. MiRNAs were extracted from exosomes derived from C166 cells and macrophages. High-throughput sequencing was then used to count the number of molecules of each individual miRNA. The number of molecules of each individual miRNA is referred to as the read count.

FIG. 4C is a series of bar graphs showing the results of an evaluation of miR combo-based cardiac reprogramming/cardiac gene expression. Cardiac fibroblasts were transfected with negmiR, miR combo or miR combo plus one of the miRNAs identified in panel A and B. After 24 hours, transfection complexes were removed. Cardiomyocyte gene expression was assessed by qPCR 14-days after the removal of transfection complexes. Expression values were normalized to the housekeeping gene Gapdh and are expressed as a fold change to the control negmiR. N=3. * P<0.05 comparisons made to miR combo.

FIG. 4D is a series of bar graphs showing the results of an evaluation of miR combo-based cardiac reprogramming/cardiac gene expression. Exosomes were isolated from 1-million C166 cells and transfected with either negmiR or miR combo alone or in combination with the anti-miR-148a-3p. Following miRNA loading, exosomes were incubated with cardiac fibroblasts for 24 hours. After 24 hours, the cell layer washed repeatedly to remove free exosomes and the cells cultured in growth media. By way of a positive control, cardiac fibroblasts were transfected with negmiR and miR combo via the standard lipid based approach. Transfection complexes were removed after 24 hours. Cardiomyocyte gene expression was assessed by qPCR 14-days after the removal of exosomes and transfection complexes. Expression values were normalized to the housekeeping gene Gapdh and are expressed as a fold change to the control negmiR. N=3. ** P<0.01, *P<0.05 comparisons made to cells transfected with miR combo via the lipid based method.

FIGS. 4A-D collectively demonstrate that the effects of C166-derived exosomes are dependent upon miR-148a-3p.

FIG. 5A is a bar graph showing MDFIC expression. Cardiac fibroblasts were transfected with negmiR or miR combo, alone or in combination with miR-148a-3p. After 24 hours, transfection complexes were removed. MDFIC gene expression was assessed by qPCR 3-days after the removal of transfection complexes. Expression values were normalized to the housekeeping gene Gapdh and are expressed as a fold change to the control negmiR. N=3. *P<0.05 comparisons made to negmiR.

FIG. 5B is a photograph of an immunoblot membrane. MDFIC expression levels were determined by immunoblotting. Protein extracts were isolated from cardiac fibroblasts 3-days after transfection with negmiR, miR combo, miR combo+miR-148a-3p, or miR-148a-3p alone. Gapdh was used as a loading control. Representative images shown. N=3.

FIG. 5C is a bar graph showing miR-148a-3p binding. Cardiac fibroblasts were transfected with a plasmid containing the MDFIC 3′UTR coupled to the Gluc/Seap reporter. Three days after transfection with the MDFIC 3′UTR reporter plasmid, the cells were further transfected with a non-targeting control miR or miR-148a-3p. Twenty-four hours later, Gluc/Seap fluorescence was measured. N=3. ** P<0.01.

FIG. 5D is a series of bar graphs showing the results of an evaluation of cell phenotype/cell marker expression. Cardiac fibroblasts and cardiomyocytes were isolated from 8-week old C57BL6 mice. Expression levels of cardiomyocyte markers (Myh6, Actn2), fibroblast markers (Col1a2, Ddr2) and MDFIC were determined by qPCR and normalized to Gapdh. N=5. *** P<0.001.

FIG. 5E is a photograph of immunostained cells. MDFIC immunostaining (green) in cardiac fibroblasts. DAPI (blue) was used as a nuclear stain. N=3. Representative images shown. Scale bar for main image: 50 micron. Scale bar for insets: 20 micron.

FIG. 5F is a bar graph showing the results of a ChIP analysis of MDFIC binding. Chromatin was isolated from cardiac fibroblasts and incubated with MDFIC or isotype control antibodies. After washing, immunoprecipitates were analyzed for the indicated cardiomyocyte and non-cardiomyocyte genes. N=3. Representative experiment shown.

FIG. 5G is a bar graph showing the results of an evaluation of MDFIC mRNA expression. Cardiac fibroblasts were transfected with a non-targeting siRNA or a siRNA which targets MDFIC. Expression levels of MDFIC were determined 3-days after transfection and normalized to the housekeeping gene Gapdh. Normalized expression values are expressed as a fold change compared to non-targeting siRNA transfected cells. N=4. *** P<0.001

FIG. 5H is a series of bar graphs showing the results of a gene expression analysis. Exosomes were isolated from 1-million C166 cells and transfected with miRNA (negmiR or miR combo) and siRNA (non-targeting or MDFIC targeting). Following loading, exosomes were incubated with cardiac fibroblasts for 24 hours. After 24 hours, the cell layer washed repeatedly to remove free exosomes and the cells cultured in growth media. Transfection complexes were removed after 24 hours. Cardiomyocyte gene expression (left panel) and non-cardiomyocyte gene expression (right panel) was assessed by qPCR 14-days after the removal of exosomes. Expression values were normalized to the housekeeping gene Gapdh and are expressed as a fold change to the control group (negmiR plus non-targeting siRNA). N=3. ** P<0.01, *P<0.05, ns—not significant: comparisons made between miR combo plus non-targeting siRNA and miR combo plus MDFIC targeting siRNA groups.

FIGS. 5A-H collectively demonstrate that miR-148a-3p mediates its effects via the transcription factor MDFIC. miR-148a-3p mediates its effects through down-modulation of the transcription factor MDFIC.

### DETAILED DESCRIPTION

Cardiac injury results in the irreversible and permanent loss of cardiomyocytes. Replenishing the cardiomyocyte population still eludes medical science. We demonstrate herein that fibroblasts within cardiac scar tissue are reprogrammed into cardiomyocytes via a set of four miRNAs, referred to as “miR combo”

miR-1:
SEQ ID NO: 1
5′UGGAAUGUAAAGAAGUAUGUAU3′,;
miR-133a-5p:
SEQ ID NO: 2
5′UUUGGUCCCCUUCAACCAGCUG3′,;
miR-208a-5p:
SEQ ID NO: 3
5′AUAAGACGAGCAAAAAGCUUGU3′,;
miR-499A-5p:
SEQ ID NO: 4
5′UUAAGACUUGCAGUGAUGUUU3′,.

Combinations of transcription factors have also been identified. Irrespective of whether miRNAs or transcription factors are utilized, the effects of fibroblast reprogramming to cardiomyocytes on cardiac function are relatively modest. The relatively modest effects are partly due to method of delivery, a problem to which the invention provides a solution.

Currently, reprogramming factors are into the heart via lenti- or retro-viruses. While viruses provided an important proof-of-principle, they suffer from significant limitations including limited packaging capacity and a lack of cell-specificity. Due to the limitations imposed by viruses, various researchers have turned to identifying alternatives.

Exosomes are small extracellular vesicles secreted by most mammalian cells. Exosomes range in size from 30 nm to 150 nm in diameter. These lipid vesicles shuttle proteins and genetic information between both neighboring and distant cells. Methods for exosome isolation are well known in the art and include techniques such as differential ultracentrifugation, size-based isolation such as ultrafiltration, membrane filtration or sieving, and or high-performance liquid chromatography (HPLC, precipitation [e.g., using polyethylene glycol (PEG)] followed by centrifugation or filtration, affinity-based capture (e.g., antibody-based or other ligand-receptor binding based), or microfluidics-based isolation methods.

By encapsulating components of their cell of origin and fusing with adjacent cells, exosomes are important mediators of cell to cell communication. Depending upon the context and the cell of origin, the proteins, mRNAs and miRNAs carried by exosomes can influence cardiac repair and regeneration in both a positive and antagonistic fashion. Through simple techniques, exosomes can also be made into cargo carriers. While cargo-carrying exosomes have been investigated as a cancer therapy, there have been no analogous studies in cardiac regeneration and regeneration.

In accordance with the invention, exosomes were made and shown to be an effective delivery system for factors, which reprogram fibroblasts into cardiomyocytes. Exosomes were isolated from a variety of different cells and assessed for their ability to deliver reprogramming factors such as miR combo into cardiac fibroblasts. Through this screening approach, exosomes derived from C166 cells were found to preferentially bind and be internalized by fibroblasts both in vitro and in vivo. Importantly, miR combo delivered by C166-derived exosomes induced cardiomyocytes (reprogramming) in vitro and in vivo. In vivo, C166 exosome mediated delivery of miR combo was associated with significant improvements in cardiac function in a myocardial infarction injury model. When compared to standard lipid based transfection methods, the C166 exosome method of delivery was found to enhance the efficacy of miR combo reprogramming. Further analysis indicated that this enhancement was due to encapsulation of the C166 miRNA miR-148a-3p into the exosome. The miR-148a-3p target was identified to be the transcription factor MDFIC and targeted knockdown of this protein enhanced the ability of miR combo to reprogram fibroblasts into cardiomyocytes.

In addition to C166 cells, other cells are useful to make exosomes suitable for delivering reprogramming factors to cardiomyocytes, e.g., to heart tissue of a human or non-human animal subject. Exemplary cells are shown in Table 1 below.

TABLE 1

| Endothelial cells | Source |
|---|---|
| Primary Umbilical Vein Endothelial Cells; | ATCC PCS-100-010 |
| Normal, Human (HUVEC) | |
| Primary Aortic Endothelial Cells; Normal, | ATCC PCS-100-011 |
| Human (HAEC) | |
| Primary Dermal Microvascular Endothelial Cells; | ATCC PCS-110-010 |
| Normal, Human, Neonatal (HDMVECn) | |
| Primary Coronary Artery Endothelial Cells; | ATCC PCS-100-020 |
| Normal, Human (HCAEC) | |
| Primary Pulmonary Artery Endothelial Cells; | ATCC PCS-100-022 |
| Normal, Human (HPAEC) | |
| C166 | ATCC CRL-2581 |
| bEnd.3 [BEND3] | ATCC CRL-2299 |
| Endothelial cells | Source |
| SVR A221a | ATCC CRL-2386 |
| SVR bag4 | ATCC CRL-2387 |
| SVEC4-10EE2 | ATCC CRL-2167 |
| SVEC4-10 | ATCC CRL-2181 |
| TIME | ATCC CRL-4025 |
| SVEC4-10EHR1 | ATCC CRL-2161 |
| EOMA | ATCC CRL-2586 |
| TeloHAEC-GFP | ATCC CRL-4054 |
| TeloHAEC | ATCC CRL-4052 |
| EJG | ATCC CRL-8659 |
| HUV-EC-C [HUVEC] | ATCC CRL-1730 |
| HUVEC/TERT 2 | ATCC CRL-4053 |
| 3B-11 | ATCC CRL-2160 |
| IP-1B | ATCC CRL-2162 |
| SK-HEP-1 | ATCC HTB-52 |
| HBEC-5i | ATCC CRL-3245 |
| HMEC-1 | ATCC CRL-3243 |
| BB19 | Rahman N A, Raisal A N H M, Meyding- |
| | Lamade U, Craemer E M, Diah S, Tuah A A, |
| | and Muharram S H. Brain Research 1642 |
| | (2016) 532-545 |
| BBEC-117 | Rahman N A, Raisal A N H M, Meyding- |
| | Lamade U, Craemer E M, Diah S, Tuah A A, |
| | and Muharram S H. Brain Research 1642 |
| | (2016) 532-545 |
| bEnd.5 | ATCC CRL-2299, Rahman N A, Raisal |
| | ANHM, Meyding-Lamade U, Craemer E M, |
| | Diah S, Tuah A A, and Muharram S H. Brain |
| | Research 1642 (2016) 532-545 |
| bEnd.WT | Rahman N A, Raisal A N H M, Meyding- |
| | Lamade U, Craemer E M, Diah S, Tuah A A, |
| | and Muharram S H. Brain Research 1642 |
| | (2016) 532-545 |
| CEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| cEND | Rahman N A, Raisal A N H M, Meyding- |
| | Lamade U, Craemer E M, Diah S, Tuah A A, |
| | and Muharram S H. Brain Research 1642 |
| | (2016) 532-545 |
| cerebEND | Rahman N A, Raisal A N H M, Meyding- |
| | Lamade U, Craemer E M, Diah S, Tuah A A, |
| | and Muharram S H. Brain Research 1642 |
| | (2016) 532-545 |
| CR3 | Rahman N A, Raisal A N H M, Meyding- |
| | Lamade U, Craemer E M, Diah S, Tuah A A, |
| | and Muharram S H. Brain Research 1642 |
| | (2016) 532-545 |
| CRL-2583 | Rahman N A, Raisal A N H M, Meyding- |
| | Lamade U, Craemer E M, Diah S, Tuah A A, |
| | and Muharram S H. Brain Research 1642 |
| | (2016) 532-545 |
| EA.hy926 | ATCC CRL-2922, |
| | Rahman N A, Raisal A N H M, Meyding- |
| | Lamade U, Craemer E M, Diah S, Tuah A A, |
| | and Muharram S H. Brain Research 1642 |
| | (2016) 532-545 |
| ECV304 | Rahman N A, Raisal A N H M, Meyding- |
| | Lamade U, Craemer E M, Diah S, Tuah A A, |
| | and Muharram S H. Brain Research 1642 |
| | (2016) 532-545 |
| GP8 | Rahman N A, Raisal A N H M, Meyding- |
| | Lamade U, Craemer E M, Diah S, Tuah A A, |
| | and Muharram S H. Brain Research 1642 |
| | (2016) 532-545 |
| GPNT | Rahman N A, Raisal A N H M, Meyding- |
| | Lamade U, Craemer E M, Diah S, Tuah A A, |
| | and Muharram S H. Brain Research 1642 |
| | (2016) 532-545 |
| HBMEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| HBMEC/ciβ | Rahman N A, Raisal A N H M, Meyding- |
| | Lamade U, Craemer E M, Diah S, Tuah A A, |
| | and Muharram S H. Brain Research 1642 |
| | (2016) 532-545 |
| HBMVEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| HCEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| hCMEC/D3 | Rahman N A, Raisal A N H M, Meyding- |
| | Lamade U, Craemer E M, Diah S, Tuah A A, |
| | and Muharram S H. Brain Research 1642 |
| | (2016) 532-545 |
| HEK 293 | ATCC CRL-1573, CRL-1573.3, |
| | Rahman N A, Raisal A N H M, Meyding- |
| | Lamade U, Craemer E M, Diah S, Tuah A A, |
| | and Muharram S H. Brain Research 1642 |
| | (2016) 532-545 |
| HUVEC-304 | Rahman N A, Raisal A N H M, Meyding- |
| | Lamade U, Craemer E M, Diah S, Tuah A A, |
| | and Muharram S H. Brain Research 1642 |
| | (2016) 532-545 |
| iHBMEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| IHEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| MBEC-4 | Rahman N A, Raisal A N H M, Meyding- |
| | Lamade U, Craemer E M, Diah S, Tuah A A, |
| | and Muharram S H. Brain Research 1642 |
| | (2016) 532-545 |
| luEnd.PECAM-1.1 | Rahman N A, Raisal A N H M, Meyding- |
| | Lamade U, Craemer E M, Diah S, Tuah A A, |
| | and Muharram S H. Brain Research 1642 |
| | (2016) 532-545 |
| NKIM-6 | Rahman N A, Raisal A N H M, Meyding- |
| | Lamade U, Craemer E M, Diah S, Tuah A A, |
| | and Muharram S H. Brain Research 1642 |
| | (2016) 532-545 |
| PBMEC/C1-2 | Rahman N A, Raisal A N H M, Meyding- |
| | Lamade U, Craemer E M, Diah S, Tuah A A, |
| | and Muharram S H. Brain Research 1642 |
| | (2016) 532-545 |
| RBCEC4 | Rahman N A, Raisal A N H M, Meyding- |
| | Lamade U, Craemer E M, Diah S, Tuah A A, |
| | and Muharram S H. Brain Research 1642 |
| | (2016) 532-545 |
| RBE4 | Rahman N A, Raisal A N H M, Meyding- |
| | Lamade U, Craemer E M, Diah S, Tuah A A, |
| | and Muharram S H. Brain Research 1642 |
| | (2016) 532-545 |
| RBEC1 | Rahman N A, Raisal A N H M, Meyding- |
| | Lamade U, Craemer E M, Diah S, Tuah A A, |
| | and Muharram S H. Brain Research 1642 |
| | (2016) 532-545 |
| THBMEC | Rahman N A, Raisal A N H M, Meyding- |
| | Lamade U, Craemer E M, Diah S, Tuah A A, |
| | and Muharram S H. Brain Research 1642 |
| | (2016) 532-545 |
| TM-BBB | Rahman N A, Raisal A N H M, Meyding- |
| | Lamade U, Craemer E M, Diah S, Tuah A A, |
| | and Muharram S H. Brain Research 1642 |
| | (2016) 532-545 |
| TR-BBB | Rahman N A, Raisal A N H M, Meyding- |
| | Lamade U, Craemer E M, Diah S, Tuah A A, |
| | and Muharram S H. Brain Research 1642 |
| | (2016) 532-545 |
| TY08 | Rahman N A, Raisal A N H M, Meyding- |
| | Lamade U, Craemer E M, Diah S, Tuah A A, |
| | and Muharram S H. Brain Research 1642 |
| | (2016) 532-545 |
| TY10 | Rahman N A, Raisal A N H M, Meyding- |
| | Lamade U, Craemer E M, Diah S, Tuah A A, |
| | and Muharram S H. Brain Research 1642 |
| | (2016) 532-545 |
| Human Dermal Lymphatic Endothelial Cells | PromoCell C-12216, C12217 |
| (HDLEC) | |
| Human Uterine Microvascular Endothelial Cells | PromoCell C-12295 |
| (HUtMEC) | |
| Human Umbilical Artery Endothelial Cells | PromoCell C-12202 |
| (HUAEC) | |
| Human Saphenous Vein Endothelial Cells | PromoCell C-12231 |
| (HSa VEC) | |
| Human Pulmonary Microvascular Endothelial | PromoCell C-12281 |
| Cells (HPMEC) | |
| Human Aortic Endothelial Cells (HAoEC) | PromoCell C-12271 |
| Human Dermal Microvascular Endothelial Cells | PromoCell C-12210, C-12212, C-12215 |
| (HDMEC) | |
| Human Cardiac Microvascular Endothelial Cells | PromoCell C-12285 |
| (HCMEC) | |

The data described herein indicates that endothelial cell derived exosomes, such as C166-derived exosomes, are an effective tool for the delivery of reprogramming factors into the heart. Moreover, a specific miRNA (miR-148a-3p) was found to enhance cardiac reprogramming efficacy.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition (e.g., a cardiac disorder, ischemic or reperfusion-related injury, etc.) manifested by a patient or to which a patient may be susceptible. The aim of treatment includes the alleviation or prevention of symptoms, slowing or stopping the progression or worsening of a disease, disorder, or condition and/or the remission of the disease, disorder or condition (e.g., a cardiac disorder, ischemic or reperfusion-related injury, etc.). As used herein, the terms “prevent,” “preventing,” “prevention,” “prophylactic treatment” and the like refer to reducing the probability of developing a disease, disorder or condition (e.g., a cardiac disorder, ischemic or reperfusion-related injury, etc.) in a subject, who does not have, but is at risk of or susceptible to developing a disease, disorder or condition.

The term “effective amount” or “therapeutically effective amount” refers to an amount sufficient to effect beneficial or desirable biological and/or clinical results.

As used herein, the term “administering” an agent, such as a therapeutic entity (e.g., a loaded exosome as provided herein) to an animal, such as a human subject or other mammal, such as a pig, or a cell, is intended to refer to dispensing, delivering or applying the agent to the intended target. In terms of the therapeutic agent, the term “administering” is intended to refer to contacting or dispensing, delivering or applying the therapeutic agent to a subject by any suitable route for delivery of the therapeutic agent to the desired location in the animal, such as a human or other mammal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

“Contacting” or “introducing” as used herein, e.g., as in “contacting a sample” or “introducing to a cell” refers to contacting/introducing a loaded exosome as provided herein directly or indirectly in vitro, ex vivo, or in vivo to a cell or subject. Contacting a sample may include addition of a compound to a sample (e.g., introducing a loaded exosome to a fibroblast), or administration to a subject. Contacting encompasses administration to a solution, cell, tissue, mammal, subject, patient, or human. Further, contacting a cell also includes adding an agent (e.g., a loaded exosome) to a cell culture.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, pigs, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

### A. Exosomes

Exosomes are membrane-bound extracellular vesicles (EVs) that are produced in the endosomal compartment of most eukaryotic cells. The multivesicular body (MVB) is an endosome defined by intraluminal vesicles (ILVs) which bud into the endosomal lumen. The inventors have found that such exosomes are an efficient delivery method for the delivery of miRs to cells. Accordingly, one aspect of the present disclosure provides a loaded exosome comprising, consisting of, or consisting essentially of an exosome having located inside at least one miRNA. Exosomes suitable for use in the present disclosure include, but are not limited to, those isolated from a cell selected from the group consisting of endothelial cells, including cells listed in Table 1 herein, C3H10T1/2 (C3H), C166, macrophage (md), and combinations thereof.

In some embodiments, exosomes for use herein include one or more miRs. A microRNA (miR) is a small (about 22-nucleotide) RNA that is derived from larger pre-miRs. MiRs act as repressors of target mRNAs by promoting their degradation, when their sequences are perfectly complementary, or inhibiting translation when their sequences contain mismatches. Micro (mi)RNAs are emerging as important regulators of cellular differentiation, their importance underscored by the fact that they are often dysregulated during carcinogenesis. Suitable miRNAs useful for the present disclosure such as miRs comprised in exosomes, include but are not limited to, the following miRs and combination of miRs: mir1; mir133; mir138; mir206; mir208; mir126; mir1, mir133; mir1, mir138; mir1, mir206; mir1, mir208; mir133, mir138; mir133, mir206; mir133, mir208; mir138, mir206; mir138, mir208; mir206, mir208; mir1, mir138, mir208; mir1, mir206, mir208; mir138, mir206, mir208; mir1, mir133, mir206; mir1, mir133, mir208; mir1, mir138, mir206; mir133, mir138, mir208; and mir133, mir138, mir206; mir1, mir133, mir208, mir499-5p; mir1, mir133, mir206, mir499-5p, combinations thereof, and the like. Suitable miRNAs useful for the present disclosure also include miR148a-3p.

In some embodiments, exosomes for use herein are derived from an endothelial cell, such as one or more of the endothelial cells listed in Table 1 herein. In some embodiments, exosomes for use herein are derived from C166 endothelial cells. In some embodiments, exosomes for use herein include cells that comprise miR148a-3p. In some embodiments, the exosomes derived from an endothelial cell, such as a C166 cell or from a cell that comprises miR148a-3p, are loaded exosomes. In some embodiments, such loaded exosomes comprise one or more miRs, such as one or more of mir1, mir133, mir208, mir499-5p. In some embodiments, such loaded exosomes comprise mir1, mir133, mir208, and mir499-5p. In some embodiments, such loaded exosomes comprise mir1, mir133, mir208, mir499-5p and miR148a-3p.

In some embodiments, loaded exosomes herein are delivered with an inhibitor of the transcription factor MDFIC. In some aspects, the inhibitor of MDFIC expression comprises a nucleic acid, for example a miR, a siRNA, or a shRNA. In some aspects, the inhibitor of MDFIC expression comprises a nucleic acid comprising miR148a-3p. In some aspects, the inhibitor of MDFIC expression comprises an siRNA or shRNA that inhibits the expression of MDFIC. In some aspects, the loaded exosomes delivered with the inhibitor of MDFIC comprise mir1, mir133, mir208, and mir499-5p.

### B. Pharmaceutical Compositions

In another aspect, the present disclosure provides compositions comprising one or more of the loaded exosomes as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent a disease, such as preventing and/or treating an ischemic or reperfusion-related injury, restoring tissue function to fibrotic tissue in an organ, etc. the loaded exosomes described herein may be administered singly, as mixtures of one or more loaded exosomes or in mixture or combination with other agents (e.g., therapeutic agents) useful for treating such diseases, conditions, and/or the symptoms associated with such diseases and/or conditions. Such agents may include, but are not limited to, aspirin, nitrates, beta blockers, calcium channel blockers, cholesterol-lowering medications, angiotensin-converting enzyme (ACE) inhibitors, ranolazine, to name a few. The loaded exosomes may be administered in the form of loaded exosomes per se, or as pharmaceutical compositions comprising a loaded exosome.

Pharmaceutical compositions comprising the loaded exosome(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries, which facilitate processing of the loaded exosome(s) into preparations which can be used pharmaceutically.

Pharmaceutical compositions may take a form suitable for virtually any mode of administration, including, for example, topical, ocular, intravenous, oral, buccal, systemic, nasal, injection, transdermal, rectal, vaginal, intracoronary, intra-arterial, etc., or a form suitable for administration by inhalation or insufflation.

For topical administration, the loaded exosome(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active loaded exosome(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active loaded exosome(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the loaded exosome(s), as is well known. For buccal administration, the compositions may take the form of tablets or lozenges formulated in conventional manner. For rectal and vaginal routes of administration, the loaded exosome(s) may be formulated as solutions (for retention enemas) suppositories or ointments containing conventional suppository bases such as cocoa butter or other glycerides.

For nasal administration or administration by inhalation or insufflation, the loaded exosome(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the loaded exosome(s) and a suitable powder base such as lactose or starch.

For ocular administration, the loaded exosome(s) may be formulated as a solution, emulsion, suspension, etc. suitable for administration to the eye. A variety of vehicles suitable for administering compounds to the eye are known in the art.

For prolonged delivery, the loaded exosome(s) can be formulated as a depot preparation for administration by implantation or intramuscular injection. The loaded exosome(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the loaded exosome(s) for percutaneous absorption may be used. To this end, permeation enhancers may be used to facilitate transdermal penetration of the loaded exosome(s).

Alternatively, other pharmaceutical delivery systems may be employed. Liposomes and emulsions are well-known examples of delivery vehicles that may be used to deliver loaded exosome(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the loaded exosome(s). The pack may, for example, comprise metal or plastic foil, such as a blister pack. The pack or dispenser device may be accompanied by instructions for administration.

The loaded exosome(s) described herein, or compositions thereof, will generally be used in an amount effective to achieve the intended result, for example in an amount effective to treat or prevent the particular disease being treated. By therapeutic benefit it is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of loaded exosome(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular loaded exosome(s) the conversation rate and efficiency into active drug compound under the selected route of administration, etc.

Determination of an effective dosage of loaded exosome(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of loaded exosome(s) into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active loaded exosome(s), the bioavailability of the loaded exosome(s), its metabolism kinetics and other pharmacokinetic properties, the mode of administration and various other factors, discussed above. Dosage amount and interval may be adjusted individually to provide plasma levels of the loaded exosome(s) which are sufficient to maintain therapeutic or prophylactic effect. For example, the loaded exosome(s) may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of loaded exosome(s) may not be related to plasma concentration. Skilled artisans will be able to optimize effective dosages without undue experimentation.

### C. Methods

The loaded exosomes provided herein, and the pharmaceutical compositions thereof, have many uses, for example, in the reprogramming of a cell, such as a fibroblast of any tissue type, e.g., heart tissue. Reprogramming is a process by which cells change phenotype, state of differentiation, or function. For example, the cellular process governs the transformation of a somatic cell into a pluripotent stem cell. This process is exploited as a tool for creating patient-specific pluripotent cells that are useful in cell replacement therapies. In “direct reprogramming”, the differentiated state of a specialized somatic cell is directly changed from one specialized somatic cell to another to another type (e.g., endocrine cells to exocrine cells or fibroblasts to neurons or, as described herein, cardiomyocytes). This process is useful for creating patient-specific pluripotent cells for cell replacement therapies. Suitable starting populations fibroblasts of various bodily tissue types, e.g., heart, lung, liver, kidney, brain.

Optionally, fibroblasts are the starting population for reprogramming. Fibroblasts are traditionally defined as cells of mesenchymal origin that produce interstitial collagen (in contrast to myocytes that form collagen type IV as part of their basement membrane), fibroblasts also produce collagen types I, III and VI. In general, fibroblasts lack a basement membrane and tend have multiple processes or sheet-like extensions. They contain an oval nucleus (with 1 or 2 nucleoli), extensive rough endoplasmic reticulum, a prominent Golgi apparatus, and abundant cytoplasmic granular material. Specific markers are scarce; however, DDR2, Postn, Tcf21, Col1α1 and Col1α2 are useful markers. These markers are expressed in fibroblasts (and other non-cardiac cells) but not by other cardiac cells. The mesenchymal cells that form the cardiac fibroblast population are believed to be derived from two principal sources: (1) the pro-epicardial organ, and (2) the epithelial-mesenchymal transformation during the formation of cardiac valves. Differentiation to cardiac fibroblasts is regulated by programmed sequences of growth factors, including FGF and PDGF.

Accordingly, another aspect of the present disclosure provides a method for promoting the direct reprogramming of a cell into cardiomyocytic cells or tissue, the method comprising, consisting of, or consisting essentially of contact the cell with a loaded exosome as provided herein.

Suitable miRNAs useful for the aspects and embodiments of the invention are described below. Such miRs comprised in exosomes, include but are not limited to, the following miRs and combinations of miRs:
mir1;
mir133;
mir138;
mir206;
mir208;
mir126;
mir1, mir133;
mir1, mir138;
mir1, mir206;
mir1, mir208;
mir133, mir138;
mir133, mir206;
mir133, mir208;
mir138, mir206;
mir138, mir208;
mir206, mir208;
mir1, mir138, mir208;
mir1, mir206, mir208;
mir138, mir206, mir208;
mir1, mir133, mir206;
mir1, mir133, mir208;
mir1, mir138, mir206;
mir133, mir138, mir208;
mir133, mir138, mir206;
mir1, mir133, mir208, mir499-5p;
mir1, mir133, mir206, mir499-5p, combinations thereof, and the like. Suitable miRNAs and combinations thereof useful for the present disclosure can also include miR148a-3p or MFDIC inhibitor. Loaded exosomes for use herein are derived from an endothelial cell, such as one or more of the endothelial cells listed in Table 1 herein. Exemplary exosomes are derived from C166 endothelial cells.

In some embodiments, the cell comprises cardiac fibrotic tissue, lung fibrotic tissue, kidney fibrotic tissue, liver fibrotic tissue, or brain fibrotic tissue.

Another aspect of the present disclosure provides a method of restoring tissue function to fibrotic tissue in an organ, the method comprising, consisting of, or consisting essentially of providing patient-derived fibroblasts and introducing to the fibroblasts a loaded exosome as provided herein.

In some embodiments, the patient-derived fibroblast comprises dermal fibroblasts. In other embodiments, the patient-derived fibroblasts comprise cardiac fibroblasts.

In one embodiment, the loaded exosomes are introduced ex vivo. In another embodiment, the loaded exosome are introduced in situ. In yet other embodiments, the loaded exosomes are introduced in vivo.

Another aspect of the present disclosure provides a method of preventing and/or treating an ischemic or reperfusion-related injury in a subject, the method comprising, consisting of, or consisting essentially of administering to the subject at risk of, or suffering from, the ischemic or reperfusion-related injury a therapeutically effective amount of a loaded exosome, or a pharmaceutical composition thereof, as provided herein.

In some embodiments, the subject is suffering from a cardiac disorder. Coronary disorders, also referred to as cardiac disorders, can be categorized into at least two groups. Acute coronary disorders include myocardial infarction, and chronic coronary disorders include chronic coronary ischemia, arteriosclerosis, congestive heart failure, angina, atherosclerosis, and myocardial hypertrophy. Other coronary disorders include stroke, dilated cardiomyopathy, restenosis, coronary artery disease, heart failure, arrhythmia, angina, or hypertension.

Another aspect of the present disclosure provides all that is described and illustrated herein.

The following examples are provided by way of illustration and not by way of limitation.

### Example 1: Exosome Delivery for Therapeutic Cardiac Reprogramming

The following materials and methods were used to carry out the experiments described below.

C166 cells: C166 cells are mouse endothelial cells, which are commercially available from the American Type Culture Collection (ATCC) under the cell line designation. CRL-2581™.

Mouse cardiac fibroblasts: cardiac fibroblasts were derived from 1 day old neonate C57BL6 mice and cultured according to established protocols.

Exosome isolation: C166 cells were seeded with exosome-free serum in T75 flasks at the concentration of 1×10⁶ cells per flask. Forty-eight hours later, the supernatant was collected, centrifuged at 500 g for 10 min at 4° C. to remove non-adherent cells, and filtered through a 0.22-μm filter. The filtrate was then ultra-centrifuged at 120,000 g for 70 min at 4° C. The resulting pellet contained the exosomes and was resuspended in PBS and ready for use.

Exosome based delivery in vitro: C166 exosomes were transfected with 5 nmol miRNA (negmiR, miR combo, miR-148a-3p: ThermoScientific) and/or 5 nmol siRNA (non-targeting, MDFIC targeting: Dharmacon) via the Exo-Fect Exosome Transfection Kit (System Biosciences) according to manufacturer's instructions.

Lipid based transfection in vitro: Mouse (C57BL/6) neonatal cardiac fibroblasts were isolated from 2 day old mouse neonates according to known methods. Following isolation fibroblasts were cultured in growth media containing DMEM (ATCC, Catalogue number 30-2002) supplemented with 15% v/v FBS (Thermo Scientific Hyclone Fetal bovine serum, Catalogue number SH30071.03, Lot number AXK49952) and 1% v/v penicillin/streptomycin (Gibco, Catalogue number 15140-122, 100 units Penicillin, 100 μg/ml Streptomycin). Fibroblasts were passaged once the cells had reached 70-80% confluence using 0.05% w/v trypsin (Gibco, Catalogue number 25300-054). Freshly isolated fibroblasts were labelled as Passage 0. Experiments were conducted with cells at passage 2. For all experiments, cells were seeded at 5000 cells/cm² in growth media. After 24 hours, the cells were transfected with 5 nmol miRNA (negmiR, miR combo, miR-148a-3p: ThermoScientific) and/or 5 nmol siRNA (non-targeting, MDFIC targeting: Dharmacon) via the lipid-based transfection reagent Dharmafect-I (ThermoScientific) according to manufacturer's instructions. Transfection complexes were removed after 24 hours and cells cultured in growth media for the duration of the experiment.

QPCR: Total RNA was extracted using Quick-RNA MiniPrep Kit according to the manufacturer's instructions (Zymo Research). Total RNA (50 ng-100 ng) was converted to cDNA using a high capacity cDNA reverse transcription kit (Applied Biosystems). cDNA was used in a standard qPCR reaction involving FAM conjugated gene specific primers (ThermoFisher) and TaqMan Gene Expression Master Mix (ThermoFisher). Primers were acquired from ThermoFisher and the assay ID numbers are: Actn2 Mm00473657_m1; Gapdh Mm99999915_m1; Myh6 Mm00440359_m1; Tnni3 Mm00437164 m1.

MiRNA-seq: miRNAs from bone marrow macrophages or C166 cells (1 million cells) were extracted via a Total Exosome RNA & Protein Isolation Kit (ThermoFisher) according to the manufacturer's instructions. Isolated miRNAs were submitted for high-throughput sequencing as 50 bp pair-end reads with a sequencing depth of 10 million total reads. Crispr-Cas9 gene-editing: Sense and anti-sense strands for the Drosha gRNAs (see below) were re-suspended in water (100 μM).

The gRNA sequences are as follows:
Drosha Exon-4 Sense: 5′CAC CGT GTC GTA CCA CCC AGG ACG A3′
(SEQ ID NO:5; NCBI Entrez Gene: 29102 Ensembl: ENSG00000113360)
Drosha Exon-4 Antisense: 5′AAA CTC GTC CTG GGT ACG ACA C3′
(SEQ ID NO:6; NCBI Entrez Gene: 29102 Ensembl: ENSG00000113360)
Drosha Exon-30 Sense: 5′CAC CGG TCT TGG TTC TTA GGG CCA C3′
(SEQ ID NO:7; NCBI Entrez Gene: 29102 Ensembl: ENSG00000113360)
Drosha Exon-30 Antisense: 5′CAC CGG TCT TGG TTC TTA GGG CCA C3′
(SEQ ID NO:8; NCBI Entrez Gene: 29102 Ensembl: ENSG00000113360)

Following resuspension, the sense and antisense strands were annealed and phosphorylated. Phosphorylation of the strands (100 nmol per strand) was catalyzed by T4 polynucleotide kinase (NEB). After a 30 minute incubation at 37ºC, the enzyme was inactivated by heating the reaction at 95° C. for 5 minutes. Annealing of the two strands was then carried out by ramping the temperature down to 25° C. at 5° C. min-1. Phosphorylated and annealed Drosha Exon-4 gRNAs were then cloned into lentiCRISPR v2 (Plasmid #52961, Addgene). Drosha Exon-30 gRNAs were cloned into pLenti-U6-tdTomato-P2A-BlasR (LRT2B) (Plasmid #110854, Addgene). Lentivirus was packaged by triple transfection of 293T cells with pxPAX2, pMD2.G, and constructed gRNAs plasmids. C166 cells were subsequently infected with the lentivirus and selected by adding 2 ug/ml puromycin and 10 ug/ml blasticidin for 10 days. Red fluorescent cells were picked manually and cultured to confluence. Immunoblotting was performed to verify Drosha deletion.

Mice, Myocardial Infarction and Exosome Injection: Adult male fibroblast-specific protein 1 Cre-tandem dimer Tomato (Fsp1-Cre:tdTomato) mice were subjected to permanent ligation of the left anterior descending coronary artery using known methods. C166-derived exosomes (20 μl; derived from 1 million C166 cells) loaded with miR combo (1 nanomol) were injected at 2 sites 2 mm below the site of ligation. Equivalent volume of PBS and an equivalent volume of C166-derived exosomes containing the non-targeting miRNA negmiR were used as controls.

Immunocytochemistry: Hearts were removed and fixed in formalin. After sectioning, sections were stained with antibodies for cardiac troponin-T (Abcam) and tdTomato (Abcam). Confocal images were captured using an LSM 510 Meta DuoScan microscope (Zeiss) and processed using LSM 5 software, version 4.2.

Fibrosis measurements: Hearts were removed and fixed in formalin. After sectioning, serial sections at 200 micron intervals through the infarct zone were stained with Masson's Trichrome. Images were captured with a Zeiss inverted microscope and data processed with ImageJ. Fibrosis measurements are reported as the percentage area of the left ventricle.

Echocardiography: B-mode and M-mode echocardiography was carried out using standard methods.

Images: Images were processed with CorelDraw and Zeiss software (Axiovision Rel4.8 and Zen Blue).

Statistics: All statistical analysis was performed using GraphPad. T-Tests and ANOVAs were used as appropriate. For ANOVA, Bonferroni post-hoc tests were used to determine significance between groups. Individual data points and a summary bar graph of Mean±SEM is shown. A P-value of less than 0.05 was considered significant.

The experiments and results described below demonstrate that C166 derived exosomes are an effective tool for the delivery of reprogramming factors into the heart. Moreover, an miRNA which enhances reprogramming efficacy was discovered. The combination of these elements represents a significant improvement over existing methods for clinical outcomes for those diagnosed and suffering from cardiac function impairment due to the presence of scar tissue in the heart.

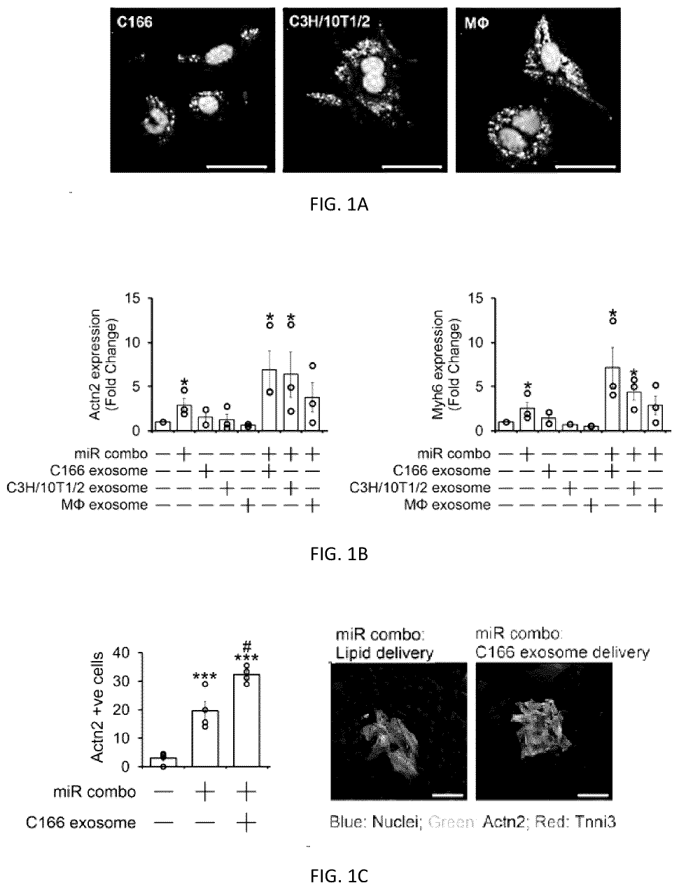

We have demonstrated that directly reprogramming scar fibroblasts into new cardiomyocytes improves cardiac function in the infarcted heart combo. Prior to the invention, a major challenge is the delivery of reprogramming factors to cardiac fibroblasts in vivo. Currently, reprogramming factors are delivered into the heart by viruses which are often poor delivery vehicles; typically lacking cell specificity and suffering from packaging size constraints. Studies were undertaken to determine whether the efficacy of direct cardiac reprogramming would be improved by optimizing delivery. To that end, we focused on identifying a fibroblast-targeting exosome. Exosomes were isolated from C166 cells, C3H/10T1/2 cells and primary macrophages and assessed for their ability to deliver a fluorescent RNA molecule into cardiac fibroblasts in vitro. As shown in FIG. 1A, exosomes from all three cell-types were capable of delivering test/model cargo, Texas-Red dye-labeled siRNA into cardiac fibroblasts. Further studies were carried to determine the applicability of exosomes as a delivery vehicle for reprogramming factors. In control cells, reprogramming was robust when the fibroblasts were transfected with miR combo via a standard lipid-based method (FIG. 1B). Both C166- and C3H/10T1/2-derived exosomes were found to be efficient delivery vehicles for miR combo as evidenced by increased mRNA levels of the cardiomyocyte proteins Actn2 and Myh6 (FIG. 1B). Based on expression levels, C166-derived exosomes were the most efficient (FIG. 1B). Macrophage-derived exosomes were found to be unsuitable for miR combo delivery as no reprogramming was observed (FIG. 1B). To verify that changes in mRNA levels were reflected in reprogramming events, cells were stained with Actn2 antibodies and the number of Actn2 cells (i.e. cardiomyocytes) were counted. As expected, miR combo transfected into cardiac fibroblasts via the standard lipid based approach gave rise to significant numbers of cardiomyocytes (FIG. 1C). Similarly, miR combo delivered into cardiac fibroblasts via C166-derived exosomes also induced the cells to reprogram to cardiomyocytes (FIG. 1C). Interestingly, the number of reprogramming events were 50% higher with the C166-derived exosome delivery route despite equivalent levels of miRNAs being delivered into the cells (FIG. 1C). This was a surprising result and indicated that the exosomes contained an agent (protein, miRNA, mRNA) that was potentiating the ability of miR combo to reprogram fibroblasts into cardiomyocytes.

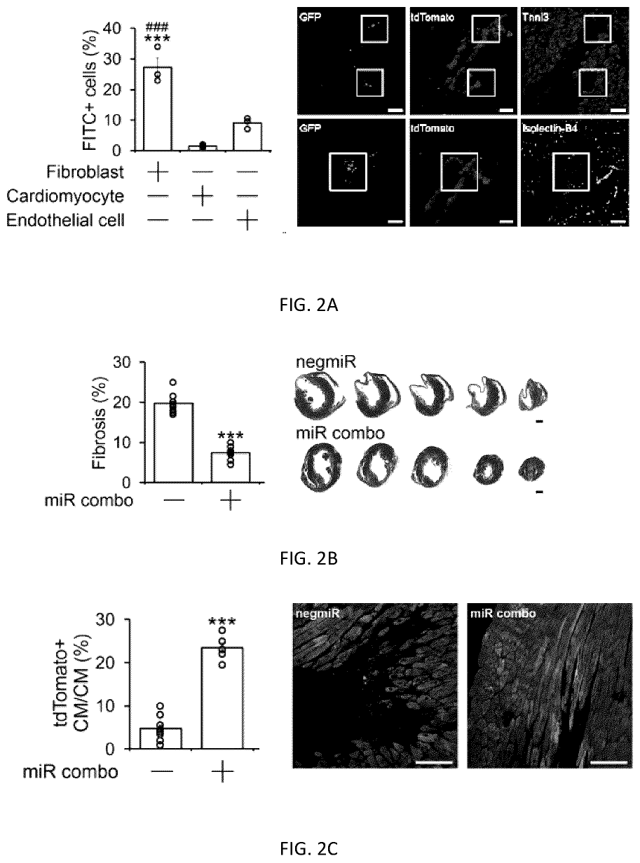

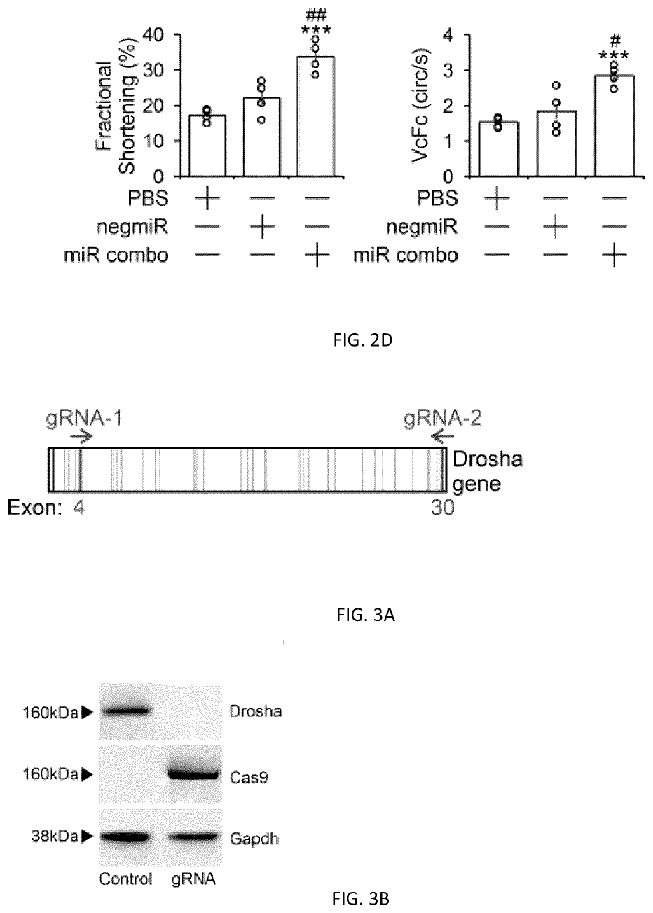

Having shown that C166 exosomes were an effective delivery vehicle for miR combo in vitro, we wanted to determine if the same was true in vivo. Firstly, we carried out experiments to determine the cell specificity of C166-derived exosomes. Consequently, C166-derived exosomes were transfected with a FITC labeled non-targeting siRNA and the loaded exosomes injected into the heart of Fsp1-Cre:tdTomato mice. In these mice, fibroblasts are permanently labeled with the fluorescent protein tdTomato. The emission spectra of FITC and tdTomato differ significantly and the two signals do not interfere with each other. Analysis of cardiac tissue for FITC and tdTomato signals, as well as cardiomyocyte-specific (Tnni3) and endothelial-specific (Isolectin-B4) markers, indicated that C166-derived exosomes displayed a significant specificity towards cardiac fibroblasts (FIG. 2A). While ˜30% of the fibroblast (tdTomato+) population were found to contain the FITC labeled siRNA, only ˜2% of cardiomyocytes and ˜8% of endothelial cells were similarly labeled (FIG. 2A).

Seeing as C166-derived exosomes were effective in delivering cargoes to fibroblasts in vivo, determined the effects on cardiac repair and regeneration when miR combo was delivered into cardiac fibroblasts by C166-derived exosomes. Consequently, C166-derived exosomes were loaded with either the non-targeting control miRNA negmiR or miR combo and then injected into heart of Fsp1-Cre:tdTomato mice immediately following MI. For human applications, exosomes are delivered into the myocardium by catheter, needle delivery, or antegrade intracoronary infusion. Exosomes bind to proteins on the cell surface, whereupon they are internalized. Thus, the specificity of C166-derived exosomes for binding to fibroblasts is due to a unique fibroblast protein not found on any other cell. Surface proteins do not differ substantially between fibroblasts of different mammalian species. Consequently, C166 derived exosomes are suitable for human therapy. Fibrosis levels were markedly attenuated in mice receiving miR combo (FIG. 2B). Reduced fibrosis was associated with the appearance of significant numbers of cardiomyocytes derived from the reprogramming of fibroblasts (FIG. 2C).

Similarly, miR combo delivery via C166-derived exosomes improved cardiac function such as fractional shortening. Fractional shortening is the reduction of the length of the end-diastolic diameter that occurs by the end of systole. Like the ejection fraction, it is a measure of the heart's muscular contractility. If the diameter fails to shorten by at least 28%, the efficiency of the heart in ejecting blood is impaired. Indeed, fractional shortening increased by 20% (FIG. 2D). This degree of recovery was greater than can be observed using viral based delivery methods.

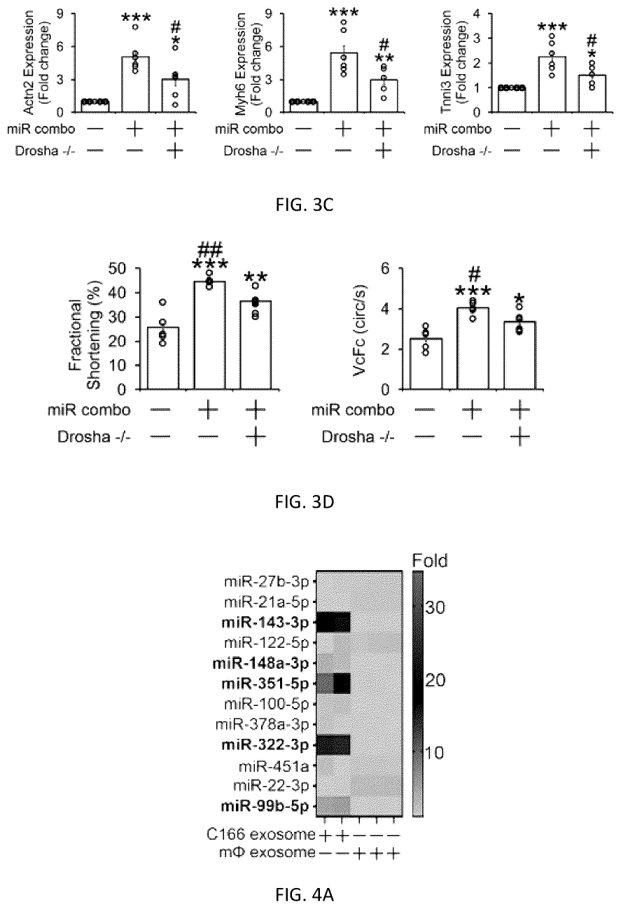

In both in vitro and in vivo models, C166-derived exosome delivery was associated with enhanced reprogramming efficacy. To determine whether this enhancement was due to a miRNA from the C166 cells being incorporated into the C166 exosomes, Drosha was targeted. Drosha is a key enzyme in miRNA processing. Ablation of Drosha expression would prevent miRNA processing and stop miRNAs from C166 cells from entering C166 exosomes. Drosha gene ablation was carried out by Cas9 gene-editing. Guide-RNAs were targeted to exon4 and exon30 of the Drosha gene and expressed in C166 cells alongside Cas9 (FIG. 3A). Gene-editing completely ablated Drosha expression from the C166 cells (FIG. 3B). To test the effects of C166 endogenous miRNAs on reprogramming efficacy, miR combo was loaded into control C166-derived exosomes and exosomes isolated from Drosha gene-edited C166 cells. Once the exosomes were loaded with miR combo, they were incubated with cardiac fibroblasts. Analysis of cardiomyocyte mRNA levels indicated that miR combo delivery via control C166 exosomes robustly reprogrammed cardiac fibroblasts (FIG. 3C). Reprogramming was attenuated when miR combo was delivered via exosomes isolated from Drosha gene-edited C166 cells (FIG. 3C). To determine the effect of C166 miRNAs in a cardiac injury model, miR combo was again loaded into control C166 exosomes and exosomes derived from Drosha gene-edited C166 cells. Once miR combo was loaded into the exosomes, the particles were injected into Fsp1-Cre:tdTomato mice immediately following MI. As shown earlier, when compared to mice receiving the control non-targeting miRNA via C166-derived exosomes, miR combo delivery via C166-derived exosomes was associated with significant functional recovery (FIG. 3D). In contrast, functional recovery was significantly reduced when miR combo was delivered into the infarcted heart via exosomes derived from Drosha gene-edited C166 cells (FIG. 3D).

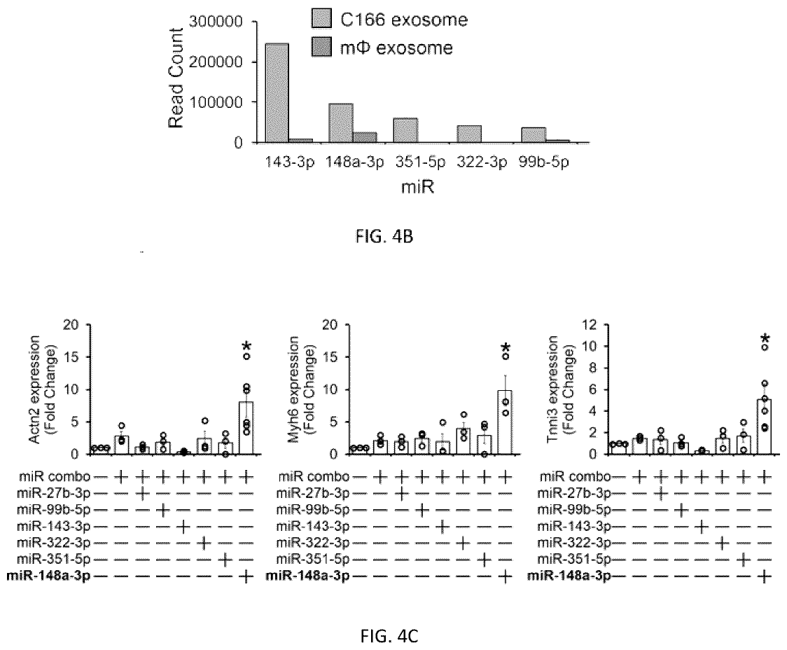

Both in vitro and in vivo experiments demonstrated that C166 cells were providing a miRNA to enhance the efficacy of miR combo-based reprogramming. To identify the C166 miRNA, we performed miRNA-seq on C166 exosomes. Potential candidate miRNAs were identified by comparing miRNAs in C166-derived exosomes with miRNAs within macrophage exosomes. Macrophage exosome miRNAs were used as the comparator, because exosomes derived from these cells were ineffective for miR combo delivery. Comparing exosome miRNA profiles identified five miRNAs which were present at significantly higher levels in C166-derived exosomes: miR-143-3p, miR-148a-3p, miR-351-5p, miR-322-3p, and miR-99b-5p (FIGS. 4A and 4B). To test the effects of these miRNAs on miR combo based reprogramming, cardiac fibroblasts were transfected with miR combo and one of the aforementioned C166 exosome miRNAs. Of the five listed miRNAs, miR-148a-3p significantly enhanced the efficacy of miR combo with respect to cardiomyocyte gene expression (FIG. 4C). To further verify that miR-148a-3p was the active C166 miRNA, C166-derived exosomes were transfected with the anti-miR-148a-3p. As shown in FIG. 4D, transfection of the C166-derived exosomes with the anti-miR-148a-3p inhibited the ability of C166-derived exosomes to enhance miR combo based reprogramming.

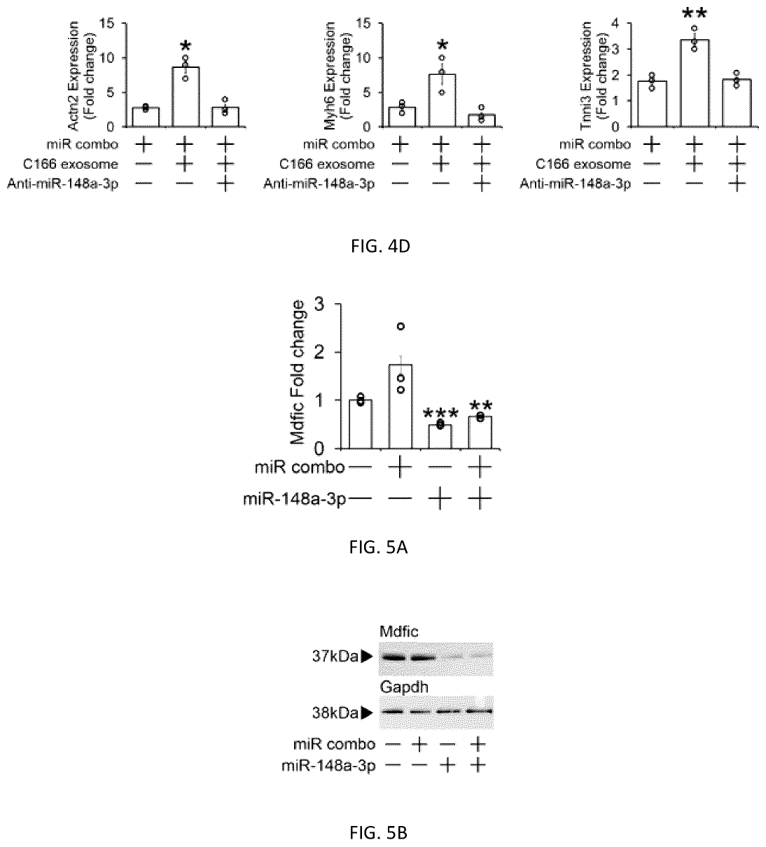

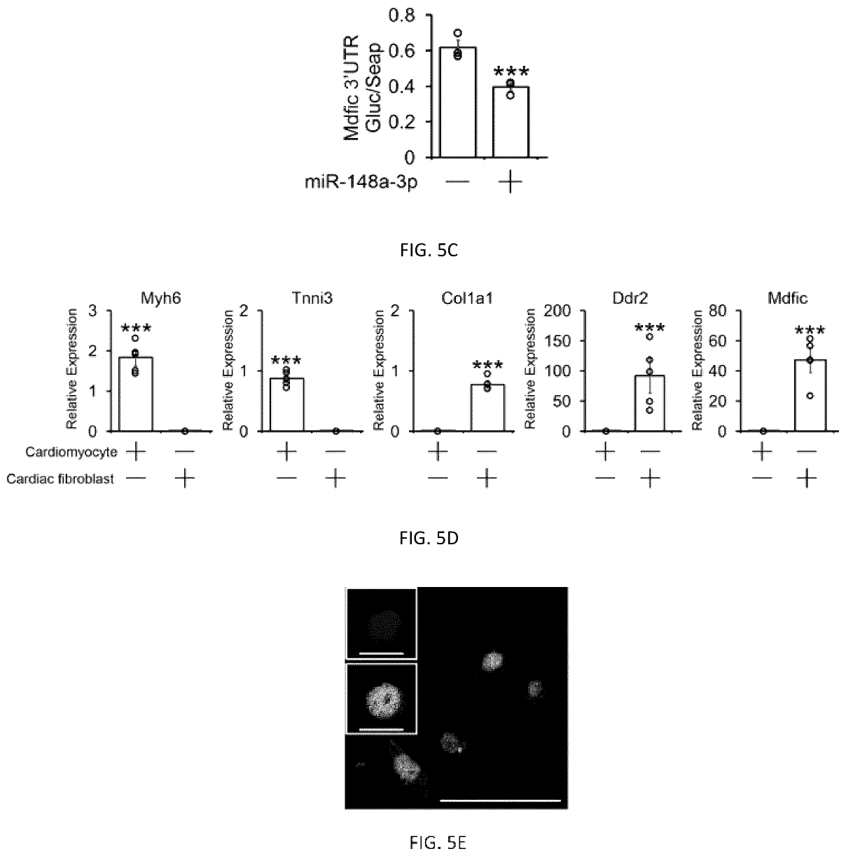

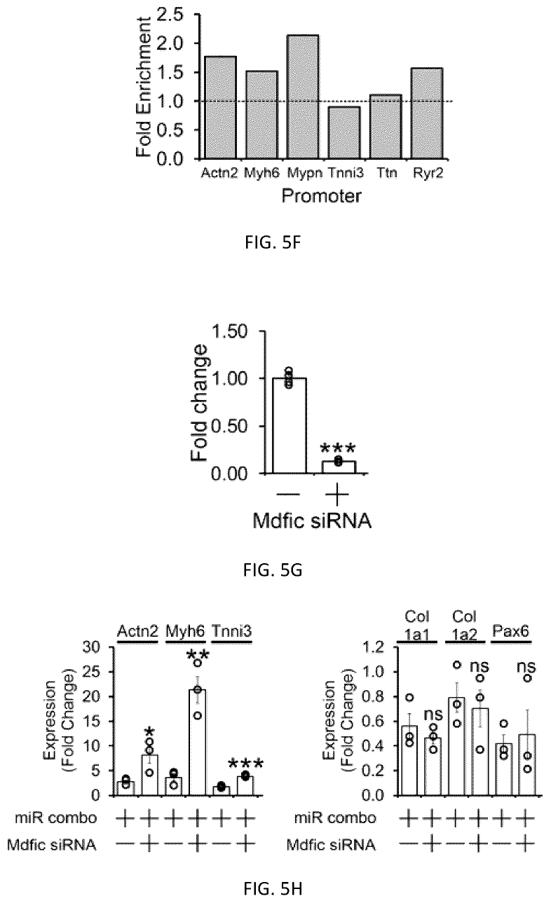

Bioinformatic analysis revealed the following possibilities as miR-148a-3p targets: Pten, Pik3r3, Rock1 and MDFIC. We chose to focus on MDFIC as this protein is related to MDFI, a known inhibitor of myogenic differentiation. Transfection studies indicated that MDFIC mRNA levels were reduced by miR-148a-3p (FIG. 5A). Interestingly, while not significant, MDFIC expression levels were increased by miR combo (FIG. 5A). Nevertheless, despite the apparent up-regulation of MDFIC levels by miR combo, the ability of miR-148a-3p to reduce MDFIC mRNA levels was undiminished by miR combo (FIG. 5A). Similar findings were observed when measuring MDFIC protein levels: MDFIC protein levels were reduced by miR-148a-3p irrespective of whether miR combo was present or not (FIG. 5B). Further studies with fluorescent reporters demonstrated that miR-148a-3p was binding to the 3′UTR of MDFIC (FIG. 5C). Taken together, these studies suggested that MDFIC was a repressor of the cardiomyocyte phenotype. To provide further evidence, we measured MDFIC mRNA levels in fibroblasts and cardiomyocytes. Cardiomyocyte and cardiac fibroblast isolations were very pure (>95% purity based on qPCR for specific markers). Cardiomyocyte isolations were significantly enriched for cardiomyocyte mRNAs (Myh6, Tnni3) and depleted for fibroblast markers (Col1a1 and Ddr2) and vice versa for cardiac fibroblast isolations (FIG. 5D). In support of a role as a repressor of the cardiomyocyte phenotype, MDFIC was found to be virtually absent in cardiomyocytes and strongly expressed in fibroblasts (FIG. 5D). To understand more about the nature of MDFIC, cardiac fibroblasts were incubated with antibodies targeting this protein. Analysis of the resulting images indicated that MDFIC resided predominantly in the nucleus (FIG. 5E). Indeed, ChIP analysis suggested that MDFIC was binding to cardiomyocyte gene promoters (FIG. 5F).

Studies were then carried out to determine the effects of reducing MDFIC expression on the efficacy of miR combo based reprogramming. A MDFIC specific siRNA strongly reduced MDFIC mRNA levels (FIG. 5G). Interestingly, the addition of MDFIC siRNA strongly enhanced the ability of miR combo to promote cardiomyocyte gene expression in fibroblasts (FIG. 5H, left panel). However, MDFIC targeting had no effect on fibroblast (Col1a1, Col1a2) or neuron (Pax6) gene expression (FIG. 5H, right panel).

### Enhanced Cardiac Reprogramming and Preferential Targeting of Cardiac Fibroblasts

A key and unexpected feature of the study was the finding that C166-derived exosomes were apparently preferentially targeting cardiac fibroblasts. This selectivity is due to a selection bias in the infarcted heart. Cardiomyocytes and endothelial cells die in the infarcted zone, while cardiac fibroblasts move into the infarcted zone. Nevertheless, our data showed that a significantly higher percentage of the fibroblast population had internalized the C166-derived exosomes when compared to the cardiomyocyte and endothelial cell populations.

The discovery of the role of miR-148a-3p in cardiomyocyte differentiation represents a significant development in the treatment of damaged or injured heart tissue. The majority of the literature suggests that miR-148a-3p influences cell behavior by modulating proliferation. Depending upon the type of cancer, miR-148a-3p either promotes or inhibits tumor progression by modulating expression of components of the cell-cycle. Similarly, miR-148a-3p induces muscle differentiation in myoblasts by inhibiting their proliferation. miR-148a-3p promotes cardiomyocyte differentiation via cell-cycle exit.

Thus, MDFIC was identified as a repressor of the cardiomyocyte phenotype. MDFIC (Human protein sequence: mrgvraataa avaataasgl srreaggrag aaaavvrppg rkcgrerrla nfpgrkrrrr rrkglgattg gcgeavsslh paphspssvr pagrrarrqr rgagsaerpm sgagealapg pvgpqrvaea gggqlgstaq gkcdkdntek ditqatnshf thgemqdqsi wgnpsdgeli rtqpqrlpql qtsaqvpsge eigkiknght glsngngihh gakhgsadnr klsapvsqkm hrkiqsslsv nsdiskkskv navfsqktgs spedccvhci laclfceflt lenivlgqas cgictseacc cccgdemgdd cncpcdmdeg imdaccessd cleicmeccg icfps (SEQ ID NO:9; HGNC: 28870 NCBI Entrez Gene: 29969 Ensembl: ENSG00000135272). Amino acids 74-246 comprise a MyoD Family Inhibitor domain), also known as HIC (human I-mfa domain-containing protein), belongs to a small family of proteins which share an 1-mfa domain.

The experiments and data described herein show that C166-derived exosomes are an effective tool for the delivery of reprogramming factors into cardiac fibroblasts. Exosome mediated delivery of reprogramming factors gave rise to fibroblast conversion into cardiomyocytes and an associated functional recovery in the infarcted heart. Furthermore, the data demonstrate that C166-derived exosomes were effective due to the presence of miR-148a-3p and the downregulation of its target protein MDFIC.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### Embodiments

The following embodiments are included in the invention.
E1. A loaded exosome comprising an exosome having located inside at least one miRNA.
E2. The loaded exosome according to claim 1 in which the exosome is isolated from a cell selected from the group consisting of C3H10T1/2 (C3H), C166, macrophage (md), and combinations thereof.
E3. The loaded exosome as in any of the preceding claims in which the miRNA is selected from the group consisting mir1; mir133; mir138; mir206; mir208; mir126; mir1, mir133; mir1, mir138; mir1, mir206; mir1, mir208; mir133, mir138; mir133, mir206; mir133, mir208; mir138, mir206; mir138, mir208; mir206, mir208; mir1, mir138, mir208; mir1, mir206, mir208; mir138, mir206, mir208; mir1, mir133, mir206; mir1, mir133, mir208; mir1, mir138, mir206; mir133, mir138, mir208; and mir133, mir138, mir206; mir1, mir133, mir208, mir499-5p; mir1, mir133, mir206, mir499-5p; and combinations thereof.
E4. A pharmaceutical composition comprising a loaded exosome as in any of the preceding claims and a pharmaceutically acceptable diluent, excipient, or carrier.
E5. A method for promoting the direct reprogramming of a cell into cardiomyocytic cells or tissue, the method comprising contacting the cell with a loaded exosome in any of E1-3 or 4.
E6. The method according to E5 in which the cell comprises cardiac fibrotic tissue.
E7. The method according to E5 in which the cell comprises a cell selected from the group consisting of fibroblasts of heart, lung, kidney, liver, or brain tissue, or combinations thereof.
E8. A method of restoring tissue function to fibrotic tissue in an organ, the method comprising providing patient-derived fibroblasts and introducing to the fibroblasts a loaded exosome as in any of E1-3 or E4.
E9. The method according to E8 in which the patient-derived fibroblast comprises dermal fibroblasts.
E10. The method according to E8 in which the patient-derived fibroblasts comprise cardiac fibroblasts.
E11. The method as in any of E5-9 or 10 in which the loaded exosomes are introduced ex vivo.
E12. The method as in any of E5-9 or 10 in which the loaded exosomes are introduced in situ.
E13. The method as in any of E5-9 or 10 in which the loaded exosomes are introduced in vivo.
E14. A method of preventing an ischemic or reperfusion-related injury in a subject, the method comprising administering to the subject at risk of the ischemic or reperfusion-related injury a therapeutically effective amount of a as in any of E1-3 or 4.
E15. A method of treating an ischemic or reperfusion-related injury in a subject, the method comprising administering to the subject suffering from the ischemic or reperfusion-related injury a therapeutically effective amount of a loaded exosome as in any of E1-3 or 4.
E16. The method according to E15 in which the subject is suffering from a cardiac disorder.
E17. All that is described and illustrated herein.
E18. Any and all methods, processes, devices, systems, devices, kits, products, materials, compositions and/or uses shown and/or described expressly or by implication in the information provided herewith, including but not limited to features that may be apparent and/or understood by those of skill in the art.
E19. A loaded exosome comprising an exosome isolated from an endothelial cell and at least one exogenous miR comprised within the exosome.
E20. The loaded exosome of E19, wherein the exosome is derived from a C166 cell.
E21. The loaded exosome of E19 or E20, wherein the exosome comprises miR148a-3p.
E22. The loaded exosome of any one of E19-21, wherein the at least one exogenous miR is selected from the group consisting of miR1, miR126, miR133, miR138, mir148a-3p, miR206, miR208, miR499-5p and any combinations thereof.
E23. The loaded exosome of claim 22, comprises 2 exogenous miR selected from the group consisting of miR1, miR126, miR133, miR138, miR206, miR208, and miR499-5p.
E24. The loaded exosome of E22, comprises 3 exogenous miR selected from the group consisting of miR1, miR 126, miR133, miR138, miR206, miR208, and miR 499-5p
E25. The loaded exosome of E22, comprises 4 exogenous miR selected from the group consisting of miR1, miR126, miR133, miR138, miR206, miR208, and miR499-5p.
E26. The loaded exosome of E22, comprises more than 4 exogenous miR selected from the group consisting of miR1, miR126, miR133, miR138, miR206, miR208, and miR499-5p.
E27. The loaded exosome of any one of E19-22, comprising miR1, miR133, miR208 and miR499-5p.
E28. A loaded exosome comprising an exosome isolated from a mammalian cell and at least one exogenous miR comprised within the exosome, wherein the at least one exogenous comprises miR miR148a-3p.
E29. The loaded exosome of E28, further comprising at least one miR selected from the group consisting of miR1, miR126, miR133, miR138, miR206, miR208, miR499-5p and any combinations thereof.
E30. The loaded exosome of E28, further comprising 2 miRs selected from the group consisting of miR1, miR126, miR133, miR138, miR206, miR208, and miR499-5p.
E31. The loaded exosome of E28, further comprising 3 miRs selected from the group consisting of miR1, miR126, miR133, miR138, miR206, miR208, and miR499-5p.
E32. The loaded exosome of E28, further comprising 4 miRs selected from the group consisting of miR1, miR126, miR133, miR138, miR206, miR208, and miR499-5p.
E33. The loaded exosome of E28, further comprising more than 4 miRs selected from the group consisting of miR1, miR126, miR133, miR138, miR206, miR208, and miR499-5p.
E34. The loaded exosome of E28, comprising miR1, miR133, miR208 and miR499-5p.
E35. An engineered cell for producing exosomes, comprising a mammalian endothelial cell and a cassette for expression of one or more exogenous miR.
E36. The engineered cell of E35, wherein the cell is a C166 cell.
E37. The engineered cell of E35, wherein the cell comprises miR148a-3p.
E38. An engineered cell for producing exosomes, comprising a mammalian cell wherein the cell comprises miR148a-3p and one or more exogenous miR.
E39. The engineered cell of any one of E35-38, wherein the at least one exogenous miR is selected from the group consisting of miR1, miR126, miR133, miR138, mir148a-3p, miR206, miR208, miR499-5p and any combinations thereof.
E40. The engineered cell of E39 comprising 2 exogenous miR selected from the group consisting of miR1, miR 126, miR133, miR 138, miR206, miR208, and miR499-5p.
E41. The engineered cell of E39 comprising 3 exogenous miR selected from the group consisting of miR1, miR126, miR133, miR138, miR206, miR208, and miR499-5p
E42. The engineered cell of E39 comprising 4 exogenous miR selected from the group consisting of miR1, miR126, miR133, miR138, miR206, miR208, and miR499-5p.
E43. The engineered cell of E39 comprising more than 4 exogenous miR selected from the group consisting of miR1, miR126, miR133, miR138, miR206, miR208, and miR499-5p.
E44. The engineered cell of E39 comprising miR1, miR133, miR208 and miR499-5p.
E45. A system for cell delivery comprising a loaded exosome and an inhibitor of MDFIC expression.
E46. The system of E45, wherein the inhibitor of MDFIC expression comprises a nucleic acid.
E47. The system of E45 or E46, wherein the nucleic acid is selected from the group consisting of miR, SiRNA, and ShRNA.
E48. The system of E47, wherein the nucleic acid comprises miR148a-3p.
E49. The system of E 47, wherein the nucleic acid comprises an siRNA or shRNA directed against MDFIC.
E50. The system of any one of E45-49, wherein the loaded exosome comprises at least one exogenous miR.
E51. The system of E50, wherein the at least one exogenous miR is selected from the group consisting of miR1, miR126, miR133, miR138, mir148a-3p, miR206, miR208, miR499-5p and any combinations thereof.
E52. The system of E50 comprising 2 exogenous miR selected from the group consisting of miR1, miR126, miR133, miR138, miR206, miR208, and miR499-5p.
E53. The system of E50 comprising 3 exogenous miR selected from the group consisting of miR1, miR126, miR133, miR138, miR206, miR208, and miR499-5p
E54. The system of E50 comprising 4 exogenous miR selected from the group consisting of miR1, miR126, miR133, miR138, miR206, miR208, and miR499-5p.
E55. The system of E50 comprising more than 4 exogenous miR selected from the group consisting of miR1, miR126, miR133, miR138, miR206, miR208, and miR499-5p.
E56. The system of E50 comprising miR1, miR133, miR208 and miR499-5p.
E57. A method of reprogramming a cell comprising administering the loaded exosome of any one of E19-34 or the system of any one of E45-56 to a cell.
E58. A method for manufacturing a loaded exosome comprising:
providing an endothelial cell comprising an inhibitor of MDFIC expression and at least one exogenous miR;
inducing the cell to produce loaded exosomes comprising the at least one exogenous miR; and
harvesting the loaded exosome.
E59. The method of E58, wherein the endothelial cell is a C166 cell.
E60. The method of E58 or E59, wherein the inhibitor of MDFIC expression comprises miR148a-3p.
E61. The method of any one of E58-60, wherein the at least one exogenous is selected from the group consisting of miR1, miR126, miR133, miR138, mir148a-3p, miR206, miR208, miR499-5p and any combinations thereof.
E62. The method of E61 comprising 2 exogenous miR selected from the group consisting of miR1, miR126, miR133, miR138, miR206, miR208, and miR499-5p.
E63. The method of E61 comprising 3 exogenous miR selected from the group consisting of miR1, miR126, miR133, miR138, miR206, miR208, and miR499-5p
E64. The method of E43 comprising 4 exogenous miR selected from the group consisting of miR1, miR126, miR133, miR138, miR206, miR208, and miR499-5p.
E65. The method of E61 comprising more than 4 exogenous miR selected from the group consisting of miR1, miR126, miR133, miR138, miR206, miR208, and miR499-5p.
E66. The method of E 61 comprising miR1, miR133, miR208 and miR499-5p.
E67. A loaded exosome manufactured by the method of any one of E57-66.
E68. A method of treating a cardiac disorder comprising delivering a loaded exosome of any one of E1-16 or E67.
E69. The method of E68, wherein the loaded exosome is delivered to a cardiac fibroblast.
E70. The method of E68, wherein the loaded exosome is delivered to a cardiac tissue in vivo.
E71. The method of E68, wherein the loaded exosome is delivered to myocardium tissue of a mammal.
E72. The method of E71, wherein the animal is a human.
E73. A method of reducing cardiac fibrosis comprising delivering a loaded exosome of any one of E19-34 or E57 to cardiac tissue in a mammal.
E74. The method of E73, wherein the delivery results in the appearance of or increase in the number of cardiomyocytes in the cardiac tissue.
E75. A method for improving cardiac function comprising delivering a loaded exosome of any one of E19-34 or E57 to cardiac tissue of a mammal.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A loaded exosome comprising an endothelial exosome, and at least one exogenous miR within said endothelial exosome.

2. The loaded exosome of claim 1, wherein the endothelial exosome is mammalian.

3. (canceled)

4. The loaded exosome of claim 1, wherein the at least one exogenous miR is selected from the group consisting of miR1, miR126, miR133, miR138, mir148a-3p, miR206, miR208, miR499-5p and any combination thereof.

5-9. (canceled)

10. The loaded exosome of claim 2, wherein said mammalian cell exosome is a C166 mammalian exosome.

11-16. (canceled)

17. An engineered cell for producing exosomes, comprising a mammalian endothelial cell and a cassette for expression of one or more exogenous miR.

18. The engineered cell of claim 17, wherein the cell is a C166 cell.

19-20. (canceled)

21. The engineered cell of claim 17, wherein the at least one exogenous miR is selected from the group consisting of miR1, miR126, miR133, miR138, mir148a-3p, miR206, miR208, miR499-5p and any combinations thereof.

22-26. (canceled)

27. A system for cell delivery comprising a loaded exosome and an inhibitor of MDFIC expression.

28. The system of claim 27, wherein the inhibitor of MDFIC expression comprises a nucleic acid.

29. The system of claim 28, wherein the nucleic acid is selected from the group consisting of miR, siRNA, and shRNA.

30-31. (canceled)

32. The system of claim 27, wherein the loaded exosome comprises at least one exogenous miR.

33. The system of claim 32, wherein the at least one exogenous miR is selected from the group consisting of miR1, miR126, miR133, miR138, mir148a-3p, miR206, miR208, miR499-5p and any combinations thereof.

34-38. (canceled)

39. A method of reprogramming a cell comprising administering the loaded exosome of claim 1 to a cell.

40. A method for manufacturing a loaded exosome comprising:
providing an endothelial cell comprising an inhibitor of MDFIC expression and at least one exogenous miR;
inducing the cell to produce loaded exosomes comprising the at least one exogenous miR; and harvesting the loaded exosome.

41. The method of claim 40, wherein the endothelial cell is a C166 cell.

42. A method of reprogramming a cell comprising administering the system of claim 27 to a cell.

43. The method of claim 40, wherein the at least one exogenous is selected from the group consisting of miR1, miR126, miR133, miR138, mir148a-3p, miR206, miR208, miR499-5p and any combinations thereof

44-48. (canceled)

49. A loaded exosome manufactured by the method of claim 40 .

50. A method of treating a cardiac disorder or reducing cardiac fibrosis or improving cardiac function comprising delivering a loaded exosome of claim 1.

51. The method of claim 50, wherein the loaded exosome is delivered to a cardiac fibroblast-or a cardiac tissue or a myocardium tissue of a mammal.

52. A method of treating cardiac disorder or reducing cardiac fibrosis or improving cardiac function comprising delivering a loaded exosome of claim 49.

53. The method of claim 52, wherein the loaded exosome is targeted or delivered to a cardiac fibroblast or a cardiac tissue or a myocardium tissue of a mammal.

54. The method of claim 51, wherein the mammal is a human.

55. The method of claim 53, wherein the mammal is a human.

56. The method of claim 50, wherein the delivery results in the appearance of or increase in the number of cardiomyocytes in the cardiac tissue.

57. The method of claim 52, wherein the delivery results in the appearance of or increase in the number of cardiomyocytes in the cardiac tissue.

58. The loaded exosome of claim 1, wherein the endothelial exosome is human endothelial exosome.
